(19) United States
(12) Patent Application Publication
(10) Pub. No.: US 2023/0131929 A1
(43) Pub. Date: Apr. 27, 2023

(54) QUINOLINE COMPOUNDS FOR TREATING RESPIRATORY DISORDERS AND VIRAL INFECTIONS

(71) Applicant: Aldeyra Therapeutics, Inc., Lexington, MA (US)

(72) Inventors: Todd BRADY, Carlisle, MA (US); Adam BROCKMAN, Arlington, MA (US); Stephen Gitu MACHATHA, Wilmington, MA (US)

(21) Appl. No.: 17/914,146

(22) PCT Filed: Mar. 24, 2021
(86) PCT No.: PCT/US2021/023884

§ 371 Date: Sep. 23, 2022

Related U.S. Application Data
(60) Provisional application No. 62/993,947, filed on Mar. 24, 2020.
(60) Provisional application No. 62/994,026, filed on Mar. 24, 2020.

(51) Int. Cl. A61K 31/47; A61P 11/00; A61P 31/14; A61K 9/00; A61K 45/06; A61P 29/00

(52) U.S. Cl. CPC A61K 31/47; A61P 11/00; A61P 31/14; A61K 9/0053; A61K 45/06; A61P 29/00

(57) ABSTRACT

The present invention relates to the use of a quinoline compound, or a pharmaceutically acceptable salt thereof, for treatment of ARDS, viral infections, and other diseases, disorders, and conditions such as those described herein.

### CROSS-REFERENCE TO RELATED APPLICATIONS

This application claims the benefit under 35 U.S.C. § 119(e) of U.S. Provisional Application Ser. No. 62/993,947, filed Mar. 24, 2020; and 62/994,026, filed Mar. 24, 2020; the entire contents of which are incorporated herein by reference.

### TECHNICAL FIELD

The present invention relates to the use of a quinoline compound, or a pharmaceutically acceptable salt thereof, for treatment of respiratory inflammatory conditions, viral infections, and other related diseases, disorders, and conditions such as those described herein.

### BACKGROUND OF THE INVENTION

Acute respiratory distress syndrome (ARDS) is a type of respiratory failure whose most salient and dangerous feature is rapid onset of widespread inflammation in the lungs. Symptoms include shortness of breath, rapid breathing, and bluish skin coloration. Among those who survive, a decreased quality of life is relatively common.

Causes may include sepsis, pancreatitis, trauma, pneumonia, and aspiration. The underlying mechanism involves diffuse injury to cells which form the barrier of the microscopic air sacs of the lungs, surfactant dysfunction, activation of the immune system, and dysfunction of the body's regulation of blood clotting. ARDS is dangerous in part because it impairs exchange of oxygen and carbon dioxide in the lungs. Diagnosis is based on a PaO₂/FiO₂ ratio (ratio of partial pressure arterial oxygen and fraction of inspired oxygen) of less than 300 mm Hg despite a positive end-expiratory pressure (PEEP) of more than 5 cm H₂O. Other causes such as heart related pulmonary edema must be excluded in order properly to diagnose ARDS.

According to the 2012 Berlin definition, ARDS is characterized by the following: lung injury of acute onset, within 1 week of an apparent clinical insult and with progression of respiratory symptoms bilateral opacities on chest imaging (chest radiograph or CT) not explained by other lung pathology (e.g., effusion, lobar/lung collapse, or nodules) respiratory failure not explained by heart failure or volume overload decreased PaO₂/FiO₂ ratio (a decreased PaO₂/FiO₂ ratio indicates reduced arterial oxygenation from the available inhaled gas): mild ARDS: 201-300 mmHg (≤39.9 kPa); moderate ARDS: 101-200 mmHg (≤26.6 kPa); severe ARDS: ≤100 mmHg (≤13.3 kPa). The Berlin definition requires a minimum positive end expiratory pressure (PEEP) of 5 cm H₂O for consideration of the PaO₂/FiO₂ ratio. This degree of PEEP may be delivered noninvasively with CPAP to diagnose mild ARDS.

The primary treatment involves mechanical ventilation together with treatments directed at the underlying cause. Ventilation strategies include using low volumes and low pressures. If oxygenation remains insufficient, lung recruitment maneuvers and neuromuscular blockers may be used. If this is insufficient, extracorporeal membrane oxygenation (ECMO) may be an option. The syndrome is associated with a death rate between 35 and 50%.

Globally, ARDS affects more than 3 million people a year. The condition was first described in 1967. Although the terminology of “adult respiratory distress syndrome” has at times been used to differentiate ARDS from “infant respiratory distress syndrome” in newborns, the international consensus is that “acute respiratory distress syndrome” is the best term because ARDS can affect people of all ages. There are modified diagnostic criteria for children and areas of the world with less resources.

ARDS can be caused by unresolved viral or bacterial infection that has caused lung inflammation and/or pneumonia. The recent global pandemic of the coronavirus disease 2019 (COVID-19) has resulted in renewed concern for the lack of effective treatments for ARDS and associated lung inflammation, sepsis, or pneumonia. Infectious diseases such as the novel viruses influenza H1N1, Middle East respiratory syndrome coronavirus (MERS-CoV), and SARS-CoV have all resulted in severe respiratory symptoms in patients.

As exemplified by COVID-19, pandemic diseases can rapidly emerge to overwhelm medical facilities worldwide with seriously and critical ill patients suffering from, among other things, pneumonia and severe respiratory difficulties associated with the virus.

Accordingly, there remains an urgent need for drugs effective for treating, preventing, and/or reducing a risk of ARDS and related diseases such as viral pneumonia and sepsis.

The threat of global pandemic of infectious diseases has become real as demonstrated by the emergence and spread of novel viruses, such as influenza H1N1, Middle East respiratory syndrome coronavirus (MERS-CoV), and coronavirus disease 2019 (COVID-19).

As exemplified by the coronavirus disease 2019 (COVID-19), this pandemic disease has rapidly emerged to overwhelm medical facilities worldwide with seriously and critical ill patients suffering from, among other things, pneumonia and severe respiratory difficulties associated with the virus. COVID-19 is an infectious disease caused by severe acute respiratory syndrome coronavirus 2 (SARS-CoV-2). The disease was first identified in 2019 in Wuhan, China, and has since spread globally, resulting in the 2019-21 coronavirus pandemic. Common symptoms include fever, cough, and shortness of breath. Additional symptoms appearing less frequently include muscle pain, sputum production, and sore throat. While the majority of cases result in mild symptoms, some progress to severe pneumonia and multi-organ failure. The lungs are the organs most affected by COVID-19 because the virus accesses host cells via the enzyme ACE2, which is most abundant in the type II alveolar cells of the lungs. The virus depends on a surface glycoprotein, “spike” (peplomer), to bind to ACE2 and enter the host cell. As the alveolar disease progresses, respiratory failure can develop, followed by death in severe cases.

SARS-CoV-2 is now at least the seventh coronavirus to show the ability to infect humans. Coroniruses have resulted in serious epidemics in the past such as MERS and SARS. The World Health Organization (WHO) declared the 2019-20 coronavirus outbreak a pandemic and a Public Health Emergency of International Concern (PHEIC). Due to reservoirs of coronavirus strains in mammals such as horseshoe bats, there remains a risk of future pandemics.

Accordingly, there remains an urgent need for drugs effective for treating, preventing, and/or reducing a risk of viral infection.

### SUMMARY OF THE INVENTION

The present invention relates to use of quinoline compounds, and pharmaceutically acceptable salts thereof and compositions thereof, for treating, preventing, and/or reducing a risk of ARDS. In one aspect of the present invention, the compounds have general formula I:

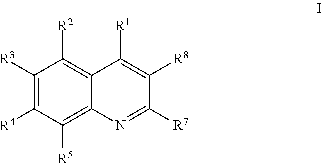

or a pharmaceutically acceptable salt thereof, wherein each variable is as defined herein.

In some embodiments, the compounds disclosed herein, or pharmaceutically acceptable salts thereof and compositions thereof, are useful for treating ARDS associated with a viral infection.

In some embodiments, a method of treating ARDS comprises administering to a patient with ARDS resulting from or associated with a viral infection an effective amount of the compound disclosed herein.

In some embodiments, the viral infection is caused by a prolate virus, helical virus, envelope virus, icosahedral virus, or a complex virus having DNA, RNA, or both DNA and RNA as its genetic material.

In some embodiments, the viral infection is caused by a coronavirus, hepatitis A virus, hepatitis B virus, dengue virus, yellow fever virus, Zika virus, influenza virus, norovirus, herpesvirus, respiratory syncytial virus (RSV), human immunodeficiency virus (HIV), Ebola virus, human T-lymphotropic virus (HTLV)-1 and -2, Epstein-Barr virus, Lassa virus, or Crimean-Congo hemorrhagic fever virus.

In some embodiments, the viral infection is caused by a coronavirus. In some embodiments, the viral infection is caused by a coronavirus selected from 229E, NL63, OC43, HKU1, MERS-CoV, SARS-CoV, and SARS-CoV-2.

In another aspect, the present invention relates to use of quinoline compounds, and pharmaceutically acceptable salts thereof and compositions thereof, for treating, preventing, and/or reducing a risk of a viral infection. In one aspect of the present invention, the compounds have general formula I, or a pharmaceutically acceptable salt thereof, wherein each variable is as defined herein.

In some embodiments, the compounds disclosed herein, or pharmaceutically acceptable salts thereof and compositions thereof, are useful for treating a viral infection, particularly viruses sensitive to quinoline compounds, such as chloroquine.

In some embodiments, a method of treating a viral infection comprises administering to a patient with a viral infection an effective amount of the compound disclosed herein.

In some embodiments, the viral infection is caused by a coronavirus, hepatitis A virus, hepatitis B virus, dengue virus, Zika virus, influenza virus, respiratory syncytial virus (RSV), human immunodeficiency virus, Ebola virua, Lassa virus, or Crimean—Congo hemorrhagic fever virus.

In some embodiments, the viral infection is caused by a coronavirus. In some embodiments, the viral infection is caused by a coronavirus selected from 229E, NL63, OC43, HKU1, MERS-CoV, SARS-CoV, and SARS-CoV-2.

### BRIEF DESCRIPTION OF THE FIGURES

FIG. 1 shows (top panel) a timeline of steps for a preclinical experiment in which NS2 (compound I-2) was compared with saline and dexamethasone (all administered IP) in an animal ARDS model. In the bottom panel, the effect of test compounds on leukocyte migration is shown in the model.

FIG. 2 shows the effect of test compounds on leukocyte migration in a preclinical ARDS model.

FIG. 3 shows the effect of test compounds on total cells, macrophages, total eosinophils, and lymphocytes in a preclinical ARDS model.

FIG. 4 and FIG. 5 show the effect of compound I-1 on levels of various inflammatory cytokines in a preclinical sepsis model. In the study, lipopolysaccharide (LPS) was administered IP to mice. 30 minutes later, I-1 was administered IP or PO. Mice were sacrificed after 2 hours, and their plasma submitted for cytokine profile analysis. IP=intraperitoneally; PO=orally; mpk=mg per kg.

### DETAILED DESCRIPTION OF THE INVENTION

### 1. General Description of Certain Aspects of the Invention

Toxic aldehydes (reactive aldehyde species, or RASP) arise during inflammatory processes in cells. It is now understood that many diseases are caused, or exacerbated, by elevated concentrations of RASP. RASP include malondialdehyde (MDA), 4-hydroxy nonenal (HNE or 4-HNE), and many others. These and other aldehydes are highly reactive to proteins, carbohydrates, lipids and DNA, leading to chemically modified biological molecules, activation of inflammatory mediators such as NF-kappaB, and damage in diverse organs. For example, retinaldehyde can react with phosphatidylethanolamine (PE) to form a highly toxic compound called A2E, which is a component of lipofuscin believed to be involved in the development and progression of Age-Related Macular Degeneration (AMD). Many bodily defense mechanisms function to remove or lower the levels of toxic aldehydes, but these are ineffective in diseases such as ARDS.

Aldehydes are implicated in inflammatory responses in the lungs. ARDS patients show significantly increased arterial levels of toxic aldehydes such as MDA and hexanal. See, e.g., Journal of Surgical Research, 168:243-253(2011). Plasma levels of HNE were also significantly elevated in ARDS patients over the course of time. See, e.g., Free Rad. Res. V21, N2, 95-106, 1994. Reducing or eliminating toxic aldehydes thus ameliorates the symptoms and treats ARDS and related pathological conditions.

Compounds described herein such as compound of Formula I, e.g., I-1 and 1-2, and pharmaceutically acceptable salts and compositions thereof, are RASP inhibitors that bind rapidly and irreversible to RASP such as 4-hydroxynonenal (4-HNE) and malondialdehyde (MDA), thus preventing RASP-mediated toxicities.

MDA, HNE and other toxic aldehydes can arise by mechanisms involving: fatty alcohols, sphingolipids, glycolipids, phytol, fatty acids, arachadonic acid metabolism, polyamine metabolism, lipid peroxidation, oxidative metabolism, and glucose metabolism. Aldehydes can cross link with primary amino groups and other chemical moieties on proteins, phospholipids, carbohydrates, and DNA, leading in many cases to toxic consequences, such as mutagenesis and carcinogenesis.

COVID-19 is an infectious disease caused by severe acute respiratory syndrome coronavirus 2 (SARS-CoV-2). The disease was first identified in 2019 in Wuhan, China, and has since spread globally, resulting in the 2019-21 coronavirus pandemic. Common symptoms include fever, cough, and shortness of breath. Although those infected with the virus may be asymptomatic, many develop flu-like symptoms including fever, cough, and shortness of breath. Emergency symptoms including difficulty breathing, persistent chest pain or pressure, confusion, difficulty waking, and bluish face or lips. Additional symptoms appearing less frequently include muscle pain, sputum production, and sore throat. While the majority of cases result in mild symptoms, some progress to severe respiratory distress, pneumonia, and/or multi-organ failure. As of Mar. 20, 2020, the rate of deaths per number of diagnosed cases was 4.1%; however, the rate of deaths ranges from 0.2% to 15% depending on age and the presence of an underlying health problem. The lungs are the organs most affected by COVID-19 because the virus accesses host cells via the enzyme ACE2, which is most abundant in the type II alveolar cells of the lungs. The virus depends on a surface glycoprotein, “spike” (peplomer), to bind to ACE2 and enter the host cell. As the alveolar disease progresses, respiratory failure can develop, followed by death in severe cases.

In certain embodiments, the present invention provides compounds, compositions, and methods for treatment, amelioration, prevention, and/or reduction of a risk of ARDS associated with a viral infection, such as COVID-19, or another described herein.

In certain embodiments, the present invention provides compounds, compositions, and method for treatment, amelioration, prevention, and/or reduction of a risk of ARDS, wherein the ARDS is associated with or caused by a direct lung insult. In some embodiments, the direct lung insult is inhalation of a substance; respiratory infection-pneumonia; aspiration of vomit; near drowning; fat pulmonary emboli; or lung transplantation.

In certain embodiments, the present invention provides compounds, compositions, and method for treatment, amelioration, prevention, and/or reduction of a risk of ARDS, wherein the ARDS is associated with or caused by an indirect lung insult. In some embodiments, the indirect lung insult is sepsis; non-respiratory infection; severe head or chest trauma; pancreatitis; drug overdose; coronary bypass; or heart-lung bypass surgery.

In some embodiments, the ARDS includes a cytokine storm. Without wishing to be bound by theory, it is believed that the presently described compounds and methods of treatment suppress such a cytokine storm while retaining patient immunity or minimizing suppression of patient immunity.

In some embodiments, the patient is co-administered a second treatment for ARDS. In some embodiments, the second treatment is mechanical (e.g., ventilator, extracorporeal membrane oxygenation (ECMO)); a steroid; an anti-cytokine; or an anticoagulant. In some embodiments, the second treatment comprises a SARS-CoV-2 treatment, such as monoclonal antibodies (e.g., bamlanivimab, casirivimab, imdevimab), remdesivir, baritimab, or convalescent plasma.

The compounds, pharmaceutically acceptable salts thereof, and compositions thereof may be combined with any of the treatments described herein and administered for treatment, amelioration, prevention, and/or reduction of a risk of ARDS associated with a direct or indirect lung insult, such as those described herein.

In one aspect, the present invention provides a method of treating ARDS, comprising administering to a patient in need thereof an effective amount of a compound of Formula I:

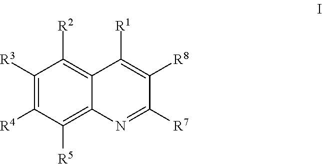

or a pharmaceutically acceptable salt thereof, wherein:
each of R¹, R⁷, and R⁸ is independently H, D, halogen, —NH₂, —CN, —OR, —SR, optionally substituted C₁₋₆ aliphatic, or

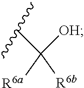

wherein one of R¹, R⁷, and R⁸ is —NH₂ and one of R¹R⁷, and R⁸ is

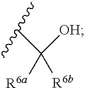

R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{6a} is C₁₋₄ aliphatic optionally substituted with 1, 2, or 3 deuterium or halogen atoms;
R^{6b} is C₁-4 aliphatic optionally substituted with 1, 2, or 3 deuterium or halogen atoms; or R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In one aspect, the present invention provides a method of treating ARDS, comprising administering to a patient in need thereof an effective amount of a compound of formula II:

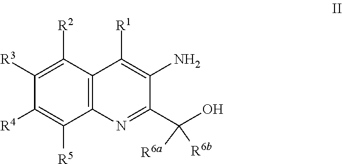

or a pharmaceutically acceptable salt thereof, wherein:
R¹ is H, D, or halogen;
R² is H, D, or halogen;
R³ is H, D, or halogen;
R⁴ is H, D, or halogen;
R⁵ is H, D, or halogen;
R^{6a} is C₁₋₄ aliphatic optionally substituted with 1, 2, or 3 deuterium or halogen atoms; and
R^{6b} is C₁₋₄ aliphatic optionally substituted with 1, 2, or 3 deuterium or halogen atoms.

In certain embodiments, the present invention provides compounds, compositions, and methods for treatment, amelioration, prevention, and/or reduction of a risk of a viral infection, such as COVID-19, or another described herein.

In one aspect, the present invention provides a method of treating a viral infection, comprising administering to a patient in need thereof an effective amount of a compound of Formula I:

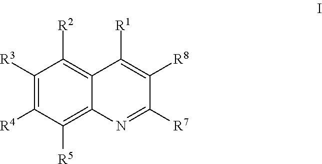

or a pharmaceutically acceptable salt thereof, wherein:
each of R¹, R⁷, and R⁸ is independently H, D, halogen, —NH₂, —CN, —OR, —SR, optionally substituted C₁₋₆ aliphatic, or

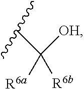

wherein one of R¹, R⁷, and R⁸ is —NH₂ and one of R¹R⁷, and R⁸ is

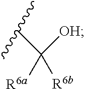

R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{6a} is C₁₋₄ aliphatic optionally substituted with 1, 2, or 3 deuterium or halogen atoms;
R^{6b} is C₁-4 aliphatic optionally substituted with 1, 2, or 3 deuterium or halogen atoms; or R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In one aspect, the present invention provides a method of treating a viral infection, comprising administering to a patient in need thereof an effective amount of a compound of formula II:

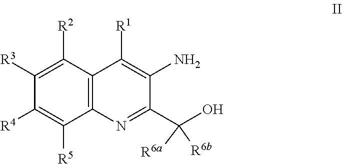

or a pharmaceutically acceptable salt thereof, wherein:
R¹ is H, D, or halogen;
R² is H, D, or halogen;
R³ is H, D, or halogen;
R⁴ is H, D, or halogen;
R⁵ is H, D, or halogen;
R^{6a} is C₁₋₄ aliphatic optionally substituted with 1, 2, or 3 deuterium or halogen atoms; and
R^{6b} is C₁₋₄ aliphatic optionally substituted with 1, 2, or 3 deuterium or halogen atoms.

### 2. Definitions

Compounds of this invention include those described generally above, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in Organic Chemistry, Thomas Sorrell, University Science Books, Sausalito: 1999, and March's Advanced Organic Chemistry, 5^{th} Ed., Ed.: Smith, M. B. and March, J., John Wiley & Sons, New York: 2001, the entire contents of which are hereby incorporated by reference.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, mesylate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

### 3. DETAILED DESCRIPTION OF EMBODIMENTS

The compounds described herein are quinoline compounds that have aldehyde trapping activity, and have been described for use in treating disorders and diseases associated with the effects of toxic aldehydes. See, e.g., PCT patent publication WO2006127945, WO2014116836, WO2017035077, and WO2017035082, each of which is hereby incorporated by reference. Synthesis of the compounds described herein are described in PCT publications WO2006127945, WO2017035082, and WO2018039192; and U.S. patent application publication US 2013/0190500, each of which is hereby incorporated by reference. As described herein, certain quinoline compounds are useful in treating ARDS, which is mediated at least in part by the presence and buildup of toxic aldehydes in the lungs.

Certain quinoline compounds, such as chloroquine and hydroxychloroquine, have been shown to have broad spectrum anti-viral activity. See, e.g., Z. Hu et al., Cell Research (2020) 30:269-271; https://doi.org/10.1038/s41422-020-0282-0. Various mechanisms of its antiviral activity have been implicated, including inhibition of pH-dependent viral entry by endocytosis, inhibition of proteolytic processing of viral proteins, and inhibition of glycosyl transferases involved in viral protein modifications. See, e.g., Savarino et al., Lancet, 2003, 3(11): 722-727).

Accordingly, in one aspect, the present invention provides a method of treating ARDS, comprising administering to a patient in need thereof an effective amount of a compound of Formula I:

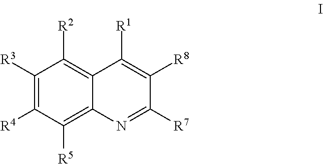

or a pharmaceutically acceptable salt thereof, wherein:
each of R¹, R⁷, and R⁸ is independently H, D, halogen, —NH₂, —CN, —OR, —SR, optionally substituted C₁₋₆ aliphatic, or

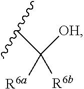

wherein one of R¹, R⁷, and R⁸ is —NH₂ and one of R¹, R⁷, and R⁸ is

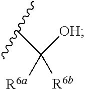

R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{6a} is C₁₋₄ aliphatic optionally substituted with 1, 2, or 3 deuterium or halogen atoms;
R^{6b} is C₁₋₄ aliphatic optionally substituted with 1, 2, or 3 deuterium or halogen atoms; or R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

In another aspect, the present invention provides a method of treating a viral infection, comprising administering to a patient in need thereof an effective amount of a compound of Formula I, or a pharmaceutically acceptable salt thereof.

In another aspect, the present invention provides a method of treating ARDS, comprising administering to a patient in need thereof an effective amount of a compound of Formula II:

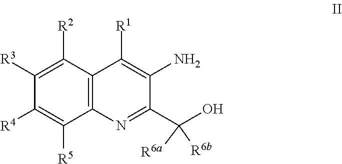

or a pharmaceutically acceptable salt thereof, wherein:
R¹ is H, D, or halogen;
R² is H, D, or halogen;
R³ is H, D, or halogen;
R⁴ is H, D, or halogen;
R⁵ is H, D, or halogen;
R^{6a} is C₁₋₄ aliphatic optionally substituted with 1, 2, or 3 deuterium or halogen atoms; and
R^{6b} is C₁₋₄ aliphatic optionally substituted with 1, 2, or 3 deuterium or halogen atoms.

In another aspect, the present invention provides a method of treating a viral infection, comprising administering to a patient in need thereof an effective amount of a compound of Formula II, or a pharmaceutically acceptable salt thereof.

The following embodiments are applicable to Formula I.

In some embodiments of Formula I, R^{6a} is C₁₋₄ aliphatic. In some embodiments, R^{6a} is C₁₋₄ aliphatic optionally substituted with 1, 2, or 3 deuterium atoms. In some embodiments, R^{6a} is C₁₋₄ aliphatic optionally substituted with 1, 2, or 3 halogen atoms.

In some embodiments of Formula I, R^{6a} is C₁₋₄ alkyl. In some embodiments, R^{6a} is C₁₋₄ alkyl optionally substituted with 1, 2, or 3 deuterium or halogen atoms. In some embodiments, R^{6a} is C₁₋₄ alkyl optionally substituted with 1, 2, or 3 halogen atoms. In some embodiments, R^{6a} is methyl or ethyl optionally substituted with 1, 2, or 3 halogen atoms. In some embodiments, R^{6a} is methyl.

As defined generally above, R^{6b} is C₁₋₄ aliphatic optionally substituted with 1, 2, or 3 deuterium or halogen atoms.

In some embodiments of Formula I, R^{6b} is C₁₋₄ aliphatic. In some embodiments, R^{6b} is C₁₋₄ aliphatic optionally substituted with 1, 2, or 3 deuterium atoms. In some embodiments, R^{6b} is C₁₋₄ aliphatic optionally substituted with 1, 2, or 3 halogen atoms.

In some embodiments of Formula I, R^{6b} is C₁₋₄ alkyl. In some embodiments, R^{6b} is C₁₋₄ alkyl optionally substituted with 1, 2, or 3 deuterium or halogen atoms. In some embodiments, R^{6b} is C₁₋₄ alkyl optionally substituted with 1, 2, or 3 halogen atoms. In some embodiments, R^{6b} is methyl or ethyl optionally substituted with 1, 2, or 3 halogen atoms. In some embodiments, R^{6b} is methyl.

As defined generally above, in some embodiments, R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur.

In some embodiments of Formula I, R^{6a} and R^{6b}, taken together with the carbon atom to which they are attached, form a 3- to 8-membered cycloalkyl. In some embodiments, R^{6a} and R^{6b}, taken together with the carbon atom to which they are attached, form a 3- to 8-membered heterocyclyl ring containing 1-2 heteroatoms selected from nitrogen, oxygen, and sulfur.

In some embodiments of Formula I, R^{6a} and R^{6b}, taken together with the carbon atom to which they are attached, form a cyclopropyl, cyclobutyl, or cyclopentyl ring. In some embodiments, R^{6a} and R^{6b}, taken together with the carbon atom to which they are attached, form an oxirane, oxetane, tetrahydrofuran, or aziridine.

In some embodiments of Formula I, the —NH₂ on one of R¹, R⁷, and R⁸ and the carbinol on the other of R¹, R⁷, and R⁸ are on adjacent carbon atoms of the pyridine moiety.

In some embodiments, the compound is a compound of Formula I-a, I-b, or I-c:

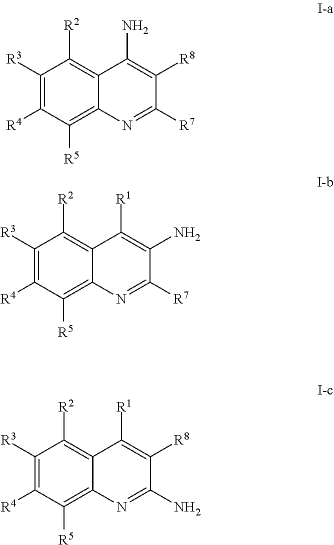

or a pharmaceutically acceptable salt thereof, wherein:
each of R¹, R⁷, and R⁸ when present is independently H, D, halogen, —CN, —OR, —SR, optionally substituted C₁₋₆ aliphatic, or

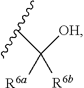

wherein one of R¹, R⁷, and R⁸ is

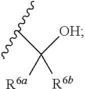

and
R², R³, R⁴, R⁵, R^{6a}, R^{6b} R⁷, R⁸ and R are as defined for Formula I.

In some embodiments, the compound for use in the method is a compound of Formula I-d, I-e, I-f, or I-g:

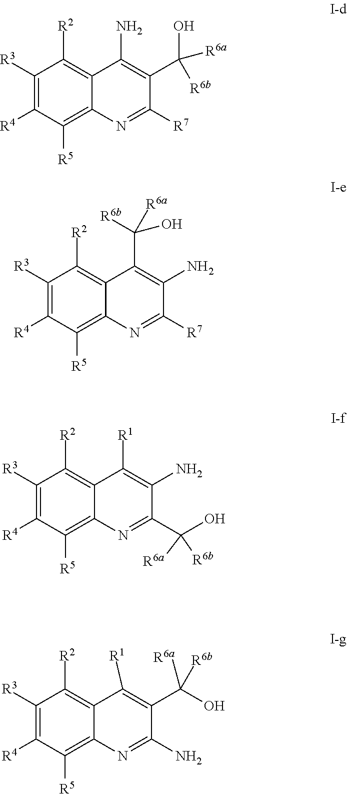

or a pharmaceutically acceptable salt thereof, wherein;
R¹ and R⁷ is independently H, D, halogen, —CN, —OR, —SR, optionally substituted C₁-6 aliphatic; and
R², R³, R⁴, R⁵, R^{6a}, R^{6b}, R⁷, R⁸ and R are as defined for Formula I.

The following embodiments are applicable to Formula II.

As defined generally above, R¹ is H, D, or halogen.

In some embodiments, R¹ is H. In some embodiments, R¹ is D. In some embodiments, R¹ is halogen. In some embodiments, R¹ is Cl. In some embodiments, R¹ is Br.

As defined generally above, R² is H, D, or halogen.

In some embodiments, R² is H. In some embodiments, R² is D. In some embodiments, R² is halogen. In some embodiments, R² is Cl. In some embodiments, R² is Br.

As defined generally above, R³ is H, D, or halogen.

In some embodiments, R³ is H. In some embodiments, R³ is D. In some embodiments, R³ is halogen. In some embodiments, R³ is Cl. In some embodiments, R³ is Br.

As defined generally above, R⁴ is H, D, or halogen.

In some embodiments, R⁴ is H. In some embodiments, R⁴ is D. In some embodiments, R⁴ is halogen. In some embodiments, R⁴ is Cl. In some embodiments, R⁴ is Br.

As defined generally above, R⁵ is H, D, or halogen.

In some embodiments, R⁵ is H. In some embodiments, R⁵ is D. In some embodiments, R⁵ is halogen. In some embodiments, R⁵ is Cl. In some embodiments, R⁵ is Br.

As defined generally above, R^{6a} is C₁₋₄ aliphatic optionally substituted with 1, 2, or 3 deuterium or halogen atoms.

In some embodiments, R^{6a} is C₁₋₄ aliphatic substituted with 1, 2, or 3 deuterium or halogen atoms. In some embodiments, R^{6a} is C₁₋₄ aliphatic. In some embodiments, R^{6a} is C₁₋₄ alkyl. In some embodiments, R^{6a} is methyl, ethyl, n-propyl, or isopropyl. In some embodiments, R^{6a} is methyl.

As defined generally above, R^{6b} is C₁₋₄ aliphatic optionally substituted with 1, 2, or 3 deuterium or halogen atoms.

In some embodiments, R^{6b} is C₁₋₄ aliphatic substituted with 1, 2, or 3 deuterium or halogen atoms. In some embodiments, R^{6b} is C₁₋₄ aliphatic. In some embodiments, R^{6b} is C₁-4 alkyl. In some embodiments, R^{6b} is C₁₋₄ alkyl optionally substituted with 1, 2, or 3 fluorine atoms. In some embodiments, R^{6b} is methyl, ethyl, n-propyl, or isopropyl. In some embodiments, R^{6b} is methyl.

In some embodiments, R^{6a} and R^{th} are methyl or ethyl. In some embodiments, R^{6a} and R^{6b} are methyl. In some embodiments, R^{6a} and R^{th} are —CD3.

In some embodiments, the compound is of Formula II-a:

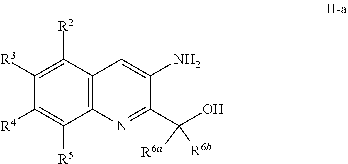

or a pharmaceutically acceptable salt thereof, wherein:
each of R², R³, R⁴, R⁵, R^{6a}, and R^{6b} is as defined is as defined above and described in embodiments herein, both singly and in combination.

In some embodiments, the compound is of Formula II-b:

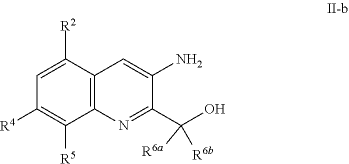

or a pharmaceutically acceptable salt thereof, wherein:
each of R², R⁴, R⁵, R^{th}, and R^{6b} is as defined is as defined above and described in embodiments herein, both singly and in combination.

In some embodiments, the compound is of any one of Formulae II-c, II-d, II-e, or II-f:

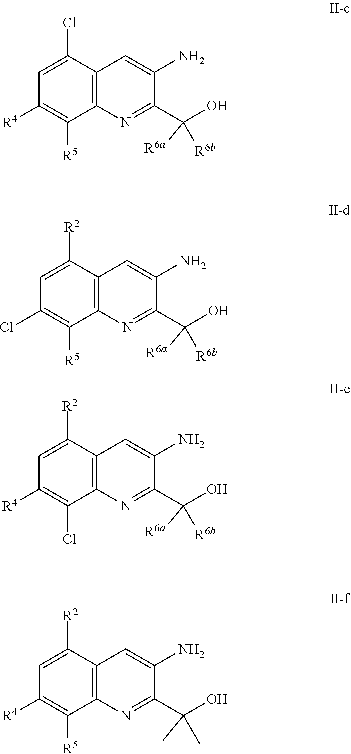

or a pharmaceutically acceptable salt thereof, wherein:
each of R², R⁴, R⁵, R^{6a}, and R^{6b} is as defined is as defined above and described in embodiments herein, both singly and in combination.

In some embodiments, the compound is of Formula II-g:

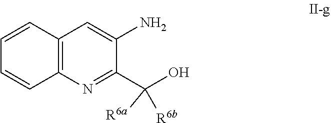

or a pharmaceutically acceptable salt thereof, wherein:
each of R^{th} and R^{6b} is as defined is as defined above and described in embodiments herein, both singly and in combination.

In some embodiments, a disclosed method comprises administering a compound selected from one depicted in Table 1, below.

TABLE 1
Representative Compounds

| | |
|---|---|
| 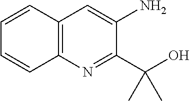 | I-1 |
| 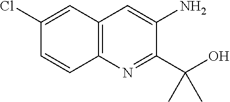 [SMILES: CC(C)(O)c1nc2ccc(Cl)cc2cc1N] | I-2 |
| 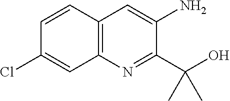 [SMILES: CC(C)(O)c1nc2cc(Cl)ccc2cc1N] | I-3 |
| 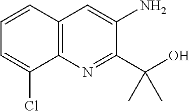 [SMILES: CC(C)(O)c1nc2c(Cl)cccc2cc1N] | I-4 |
| 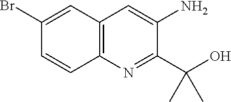 [SMILES: CC(C)(O)c1nc2ccc(Br)cc2cc1N] | I-5 |
| 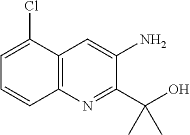 [SMILES: CC(C)(O)c1nc2cccc(Cl)c2cc1N] | I-6 |
| 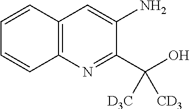 [SMILES: [2H]C([2H])([2H])C(O)(c1nc2ccccc2cc1N)C([2H])([2H])[2H]] | I-7 |
| 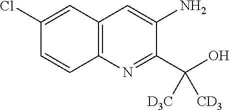 [SMILES: [2H]C([2H])([2H])C(O)(c1nc2ccc(Cl)cc2cc1N)C([2H])([2H])[2H]] | I-8 |
| 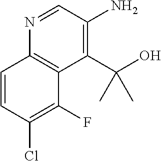 [SMILES: CC(C)(O)c1c(N)cnc2ccc(Cl)c(F)c12] | I-9 |
| 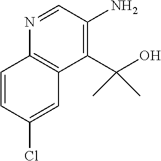 [SMILES: CC(C)(O)c1c(N)cnc2ccc(Cl)cc12] | I-10 |
| 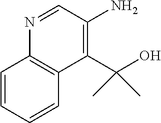 | I-11 |
| 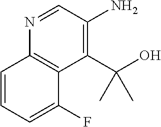 [SMILES: CC(C)(O)c1c(N)cnc2cccc(F)c12] | I-12 |
| 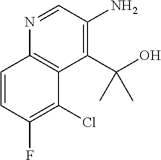 [SMILES: CC(C)(O)c1c(N)cnc2ccc(F)c(Cl)c12] | I-13 |
| 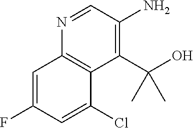 [SMILES: CC(C)(O)c1c(N)cnc2cc(F)cc(Cl)c12] | I-14 |
| 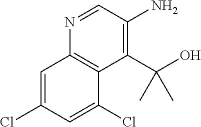 [SMILES: CC(C)(O)c1c(N)cnc2cc(Cl)cc(Cl)c12] | I-15 |
| 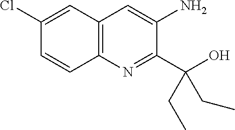 [SMILES: CCC(O)(CC)c1nc2ccc(Cl)cc2cc1N] | I-16 |
| 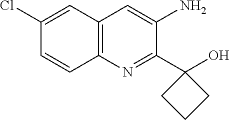 [SMILES: Nc1cc2cc(Cl)ccc2nc1C1(O)CCC1] | I-17 |
| 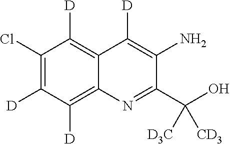 [SMILES: [2H]c1c(Cl)c([2H])c2c([2H])c(N)c(C(O)(C([2H])([2H])[2H])C([2H])([2H])[2H])nc2c1[2H]] | X-1 |
| 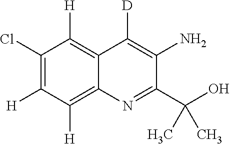 [SMILES: [2H]c1c(N)c(C(C)(C)O)nc2ccc(Cl)cc12] | X-2 |
| 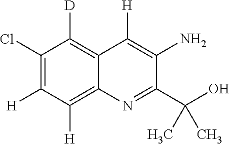 [SMILES: [2H]c1c(Cl)ccc2nc(C(C)(C)O)c(N)cc12] | X-3 |
| 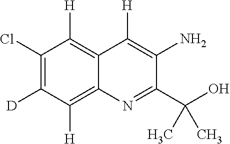 [SMILES: [2H]c1cc2nc(C(C)(C)O)c(N)cc2cc1Cl] | X-4 |
| 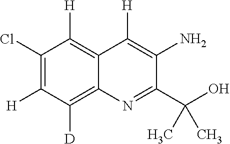 [SMILES: [2H]c1cc(Cl)cc2cc(N)c(C(C)(C)O)nc12] | X-5 |
| 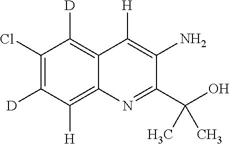 [SMILES: [2H]c1cc2nc(C(C)(C)O)c(N)cc2c([2H])c1Cl] | X-6 |
| 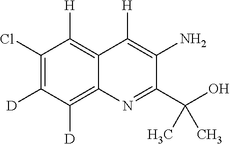 [SMILES: [2H]c1c(Cl)cc2cc(N)c(C(C)(C)O)nc2c1[2H]] | X-7 |
| 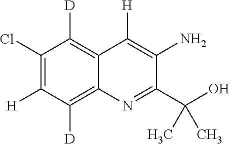 [SMILES: [2H]c1c(Cl)cc([2H])c2nc(C(C)(C)O)c(N)cc12] | X-8 |
| 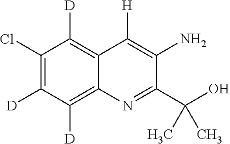 [SMILES: [2H]c1c(Cl)c([2H])c2cc(N)c(C(C)(C)O)nc2c1[2H]] | X-9 |
| 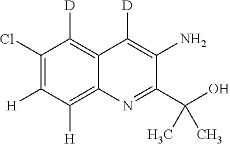 [SMILES: [2H]c1c(Cl)ccc2nc(C(C)(C)O)c(N)c([2H])c12] | X-10 |
| 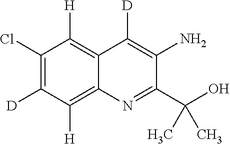 [SMILES: [2H]c1cc2nc(C(C)(C)O)c(N)c([2H])c2cc1Cl] | X-11 |
| 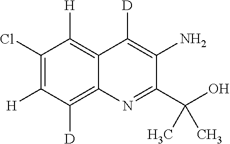 [SMILES: [2H]c1c(N)c(C(C)(C)O)nc2c([2H])cc(Cl)cc12] | X-12 |
| 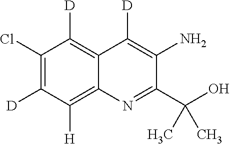 [SMILES: [2H]c1cc2nc(C(C)(C)O)c(N)c([2H])c2c([2H])c1Cl] | X-13 |
| 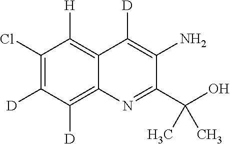 [SMILES: [2H]c1c(Cl)cc2c([2H])c(N)c(C(C)(C)O)nc2c1[2H]] | X-14 |
| 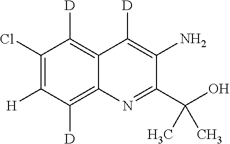 [SMILES: [2H]c1cc(Cl)c([2H])c2c([2H])c(N)c(C(C)(C)O)nc12] | X-15 |
| 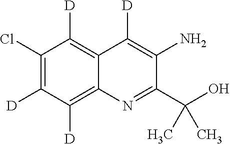 [SMILES: [2H]c1c(Cl)c([2H])c2c([2H])c(N)c(C(C)(C)O)nc2c1[2H]] | X-16 |
| 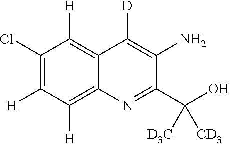 [SMILES: [2H]c1c(N)c(C(O)(C([2H])([2H])[2H])C([2H])([2H])[2H])nc2ccc(Cl)cc12] | X-17 |
| 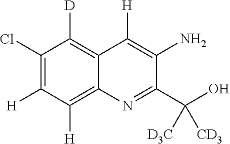 [SMILES: [2H]c1c(Cl)ccc2nc(C(O)(C([2H])([2H])[2H])C([2H])([2H])[2H])c(N)cc12] | X-18 |
| 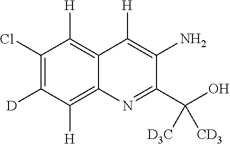 [SMILES: [2H]c1cc2nc(C(O)(C([2H])([2H])[2H])C([2H])([2H])[2H])c(N)cc2cc1Cl] | X-19 |
| 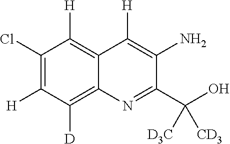 [SMILES: [2H]c1cc(Cl)cc2cc(N)c(C(O)(C([2H])([2H])[2H])C([2H])([2H])[2H])nc12] | X-20 |
| 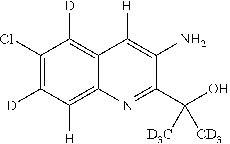 [SMILES: [2H]c1cc2nc(C(O)(C([2H])([2H])[2H])C([2H])([2H])[2H])c(N)cc2c([2H])c1Cl] | X-21 |
| 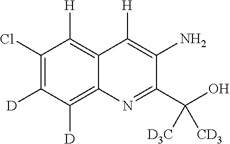 [SMILES: [2H]c1c(Cl)cc2cc(N)c(C(O)(C([2H])([2H])[2H])C([2H])([2H])[2H])nc2c1[2H]] | X-22 |
| 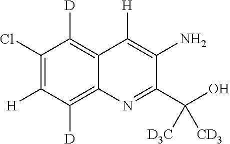 [SMILES: [2H]c1c(Cl)cc([2H])c2nc(C(O)(C([2H])([2H])[2H])C([2H])([2H])[2H])c(N)cc12] | X-23 |
| 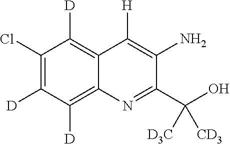 [SMILES: [2H]c1c(Cl)c([2H])c2cc(N)c(C(O)(C([2H])([2H])[2H])C([2H])([2H])[2H])nc2c1[2H]] | X-24 |
| 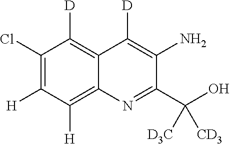 [SMILES: [2H]c1c(Cl)ccc2nc(C(O)(C([2H])([2H])[2H])C([2H])([2H])[2H])c(N)c([2H])c12] | X-25 |
| 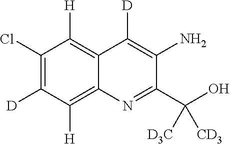 [SMILES: [2H]c1cc2nc(C(O)(C([2H])([2H])[2H])C([2H])([2H])[2H])c(N)c([2H])c2cc1Cl] | X-26 |
| 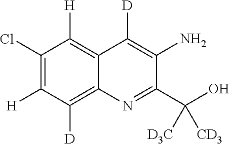 [SMILES: [2H]c1c(N)c(C(O)(C([2H])([2H])[2H])C([2H])([2H])[2H])nc2c([2H])cc(Cl)cc12] | X-27 |
| 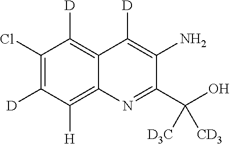 [SMILES: [2H]c1cc2nc(C(O)(C([2H])([2H])[2H])C([2H])([2H])[2H])c(N)c([2H])c2c([2H])c1Cl] | X-28 |
| 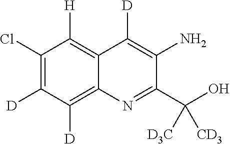 [SMILES: [2H]c1c(Cl)cc2c([2H])c(N)c(C(O)(C([2H])([2H])[2H])C([2H])([2H])[2H])nc2c1[2H]] | X-29 |
| 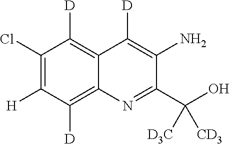 [SMILES: [2H]c1cc(Cl)c([2H])c2c([2H])c(N)c(C(O)(C([2H])([2H])[2H])C([2H])([2H])[2H])nc12] | X-30 |

In some embodiments, the present invention provides a compound depicted in Table 1, above, or a pharmaceutically acceptable salt thereof, for use in a disclosed method of treatment.

In some embodiments, the present invention provides any compound described above and herein, or a pharmaceutically acceptable salt thereof, for use in a disclosed method of treatment. As used herein, the terms “treatment,” “treat,” and “treating” refer to reversing, alleviating, delaying the onset of, or inhibiting the progress of a disease or disorder, or one or more symptoms thereof, as described herein. In some embodiments, treatment is administered after one or more symptoms have developed. In other embodiments, treatment is administered in the absence of symptoms. For example, treatment is administered to a susceptible individual prior to the onset of symptoms (e.g., in light of a history of symptoms and/or in light of genetic or other susceptibility factors). Treatment is also continued after symptoms have resolved, for example to prevent, delay or lessen the severity of their recurrence.

In some embodiments, the compounds described herein are used for the treatment, prevention, and/or reduction of a risk of ARDS.

In some embodiments, the compound described herein are used in the manufacture of a medicament for the treatment, prevention, and/or reduction of a risk of ARDS.

In some embodiments, a method of treating, preventing, and/or reducing of a risk of ARDS comprises administering an effective amount of a compound described herein.

In some embodiments, the compound is any one of the exemplary compounds of Table 1.

In some embodiments, the compound for use in treating, preventing, and/or reducing of a risk of ARDS is a compound of Formula II-g or a pharmaceutically acceptable salt thereof. In some embodiments, the compound for use in treating, preventing, and/or reducing of a risk of ARDS is compound I-1 or 1-2, or a pharmaceutically acceptable salt thereof.

In some embodiments, the ARDS is associated with or caused by a viral infection.

In some embodiments, the viral infection is accompanied by viral sepsis.

In some embodiments, the viral infection is accompanied by viral pneumonia.

In some embodiments, the viral infection is caused by a coronavirus, hepatitis A virus, hepatitis B virus, dengue virus, yellow fever virus, Zika virus, influenza virus, norovirus, herpesvirus, human immunodeficiency virus (HIV), Ebola virus, human T-lymphotropic virus (HTLV)-1 and -2, Epstein-Barr virus, Lassa virus, or Crimean-Congo hemorrhagic fever virus.

In some embodiments, the viral infection is caused by a coronavirus. In some embodiments, the coronavirus is an alpha, beta, gamma, or delta coronavirus. In some embodiments, the coronavirus is one associated with severe respiratory symptoms such as SARS.

In some embodiments, the viral infection is caused by a coronavirus, wherein the coronavirus is 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), or SARS-CoV-2 (coronavirus disease 2019, or COVID-19).

In some embodiments, the viral infection is by a respiratory syncytial virus (RSV), influenza virus, coronavirus, or herpesvirus.

In some embodiments, the viral infection is by a coronavirus.

In some embodiments, the coronavirus is selected from 229E, NL63, OC43, HKU1, MERS-CoV, SARS-CoV, and SARS-CoV-2.

In some embodiments, the viral infection is by SARS-CoV-2.

In some embodiments, the viral infection is by an influenza virus.

In some embodiments, the influenza virus is influenza type A or influenza type B.

In some embodiments, the influenza virus is B/Yamagata or B/Victoria.

In some embodiments, the influenza virus is H5N1, H1N1 or H3N2.

In some embodiments, the ARDS is associated with a bacterial infection.

In some embodiments, the bacterial infection is accompanied by bacterial sepsis.

In some embodiments, the bacterial infection is accompanied by bacterial pneumonia.

In some embodiments, the bacterial infection is by Streptococcus pneumoniae, Staphylococcus aureus, Legionella pneumophila, Pneumocystis jirovecii, or Haemophilus influenza.

In some embodiments, the ARDS is associated with acute injury to the lungs caused by a chemical toxin or physical trauma.

In some embodiments, the acute injury to the lungs is by a chemical toxin.

In some embodiments, the chemical toxin that causes acute lung injury is a choking agent, vesicant, or nerve agent.

In some embodiments, the chemical toxin is a choking agent, wherein the choking agent is chlorine gas, phosgene, carbonyl chloride, hydrogen sulfide, or ammonia.

In some embodiments, the chemical toxin is a vesicant, wherein the vesicant is sulfur mustard or nitrogen mustard.

In some embodiments, the chemical toxin is a nerve agent, wherein the nerve agent is tabun, sarin, soman, or VX.

In some embodiments, the ARDS is associated with acute injury to the lungs caused by a biological toxin.

In some embodiments, the biological toxin is ricin, botulinum toxin, or staphylococcal enterotoxin B.

In some embodiments, the patient is being treated by mechanical ventilation.

In some embodiments, the dose of the compound or pharmaceutically acceptable salt thereof is about 10 mg to about 10,000 mg per day.

In some embodiments, the dose of the compound or pharmaceutically acceptable salt thereof is about 10 mg to about 7500 mg per day.

In some embodiments, the dose of the compound or pharmaceutically acceptable salt thereof is about 50 mg to about 3600 mg per day.

In some embodiments, the dose of the compound or pharmaceutically acceptable salt thereof is about 250 mg to about 2400 mg per day.

In some embodiments, the dose of the compound or pharmaceutically acceptable salt thereof is about 600 mg to about 5000 mg per day.

In some embodiments, the dose of the compound or pharmaceutically acceptable salt thereof is about 1000 mg to about 7500 mg per day.

In some embodiments, the compound or pharmaceutically acceptable salt thereof is administered once, twice, thrice, or four times per day. In some embodiments, the compound or pharmaceutically acceptable salt thereof is administered twice per day.

In some embodiments, the dose of the compound or pharmaceutically acceptable salt thereof is about 600 mg BID (i.e., twice per day); 1.2 g BID; or 2.4 g BID.

In some embodiments, the dose of the compound or pharmaceutically acceptable salt thereof is administered systemically.

In some embodiments, the dose of the compound or pharmaceutically acceptable salt thereof is administered orally.

In some embodiments, the method further comprises administering to the patient an effective amount of a second therapeutic agent suitable for treating ARDS.

In some embodiments, the second therapeutic agent is an anti-inflammatory agent selected from a steroid, anti-GM-CSF antibody, and a lung surfactant.

In some embodiments, the compound is I-1 or a pharmaceutically acceptable salt thereof. In some embodiments, the compound is I-2 or a pharmaceutically acceptable salt thereof.

In some embodiments, the compounds described herein are used for the treatment, prevention, and/or reduction of a risk of a viral infection.

In some embodiments, the compound described herein are used in the manufacture of a medicament for the treatment, prevention, and/or reduction of a risk of a viral infection.

In some embodiments, a method of treating, preventing, and/or reducing of a risk of a viral infection, comprises administering an effective amount of a compound described herein.

In some embodiments, the compound is any one of the exemplary compounds of Table 1.

In some embodiments, the compound for use in treating, preventing, and/or reducing of a risk of a viral infection is a compound of Formula II-g or a pharmaceutically acceptable salt thereof. In some embodiments, the compound for use in treating, preventing, and/or reducing of a risk of a viral infection is compound I-1 or 1-2, or a pharmaceutically acceptable salt thereof.

In some embodiments, the viral infection is caused by a coronavirus, hepatitis A virus, hepatitis B virus, dengue virus, yellow fever virus, Zika virus, influenza virus, respiratory syncytial virus (RSV), norovirus, herpesvirus, human immunodeficiency virus (HIV), Ebola virus, human T-lymphotropic virus (HTLV)-1 and -2, Epstein-Barr virus, Lassa virus, or Crimean-Congo hemorrhagic fever virus.

In some embodiments, the viral infection is caused by a coronavirus. In some embodiments, the coronavirus is an alpha, beta, gamma, or delta coronavirus. In some embodiments, the coronavirus is one associated with severe respiratory symptoms such as SARS.

In some embodiments, the viral infection is caused by a coronavirus, wherein the coronavirus is 229E (alpha coronavirus), NL63 (alpha coronavirus), OC43 (beta coronavirus), HKU1 (beta coronavirus), MERS-CoV (the beta coronavirus that causes Middle East Respiratory Syndrome, or MERS), SARS-CoV (the beta coronavirus that causes severe acute respiratory syndrome, or SARS), or SARS-CoV-2 (coronavirus disease 2019, or COVID-19).

In some embodiments, the viral infection is caused by SARS-CoV-2.

In some embodiments, the viral infection is caused by an influenza virus.

In some embodiments, the viral infection is caused by an influenza virus, wherein the viral infection is selected from influenza type A and influenza type B. In some embodiments, the influenza virus is B/Yamagata or B/Victoria.

In some embodiments, the viral infection is caused by an influenza virus selected from H5N1, H1N1 and H3N2.

In some embodiments, the viral infection is caused by a Zika virus.

In some embodiments, the dose of the compound or pharmaceutically acceptable salt thereof is about 10 mg to about 10,000 mg per day.

In some embodiments, the dose of the compound or pharmaceutically acceptable salt thereof is about 10 mg to about 7500 mg per day.

In some embodiments, the dose of the compound or pharmaceutically acceptable salt thereof is about 50 mg to about 3600 mg per day.

In some embodiments, the dose of the compound or pharmaceutically acceptable salt thereof is about 150 mg to about 1200 mg per day.

In some embodiments, the dose of the compound or pharmaceutically acceptable salt thereof is about 400 mg to about 1200 mg per day.

In some embodiments, the dose of the compound or pharmaceutically acceptable salt thereof is about 500 mg to about 1000 mg per day.

In some embodiments, the dose of the compound or pharmaceutically acceptable salt thereof is about 600 mg to about 1200 mg per day.

In some embodiments, the dose of the compound or pharmaceutically acceptable salt thereof is about 250 mg to about 2400 mg per day.

In some embodiments, the dose of the compound or pharmaceutically acceptable salt thereof is about 600 mg to about 5000 mg per day.

In some embodiments, the dose of the compound or pharmaceutically acceptable salt thereof is about 1000 mg to about 7500 mg per day.

In some embodiments, the compound or pharmaceutically acceptable salt thereof is administered once, twice, thrice, or four times per day. In some embodiments, the compound or pharmaceutically acceptable salt thereof is administered twice per day.

In some embodiments, the dose of the compound or pharmaceutically acceptable salt thereof is about 600 mg BID (i.e., twice per day); 1.2 g BID; or 2.4 g BID.

In some embodiments, the dose of the compound or pharmaceutically acceptable salt thereof is about 200 mg BID, 300 mg BID, 400 mg BID, 500 mg BID, 600 mg BID, 700 mg BID, or 800 mg BID.

In some embodiments, the dose of the compound or pharmaceutically acceptable salt thereof is about 300 mg BID.

In some embodiments, the dose of the compound or pharmaceutically acceptable salt thereof is administered systemically.

In some embodiments, the dose of the compound or pharmaceutically acceptable salt thereof is administered orally.

In some embodiments, the dose of the compound or pharmaceutically acceptable salt thereof is administered to the patient in a fasted state. In some embodiments, the patient has consumed no food for at least 2 hours prior to dosing and at least 1 hour after dosing.

In some embodiments, the compound is I-1 or a pharmaceutically acceptable salt thereof. In some embodiments, the compound is I-2 or a pharmaceutically acceptable salt thereof.

In some embodiments, the compound reduces systemic inflammation in the patient.

In some embodiments, the compound reduces plasma levels of a biomarker selected from IL-10, IL-6, IL-10, and tumor necrosis factor alpha. In some embodiments, the compound reduces plasma levels of a biomarker selected from a RASP. In some embodiments, the RASP is malondialdehyde (MDA) and/or 4-hydroxynonenal (4-HNE).

In some embodiments, the patient has been diagnosed with asymptomatic or mild COVID-19. In some embodiments, the patient has been diagnosed with moderate COVID-19. In some embodiments, the patient has been diagnosed with severe COVID-19. In some embodiments, the patient has been diagnosed with critical COVID-19 (the patient is in critical condition).

### 4. Pharmaceutical Compositions, Administration, and Dosages

The compounds and compositions, according to the methods of the present invention, are administered using any amount and any route of administration effective for treating or lessening the severity of a disease provided above. The exact amount required will vary from subject to subject, depending on the species, age, and general condition of the subject, the severity of the infection, the particular agent, its mode of administration, and the like. Compounds of the invention are preferably formulated in unit dosage form for ease of administration and uniformity of dosage. The expression “unit dosage form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), buccally, as an oral or nasal spray, or the like, depending on the severity of the disease being treated. In certain embodiments, the compounds of the invention are administered orally or parenterally at dosage levels of about 0.01 mg/kg to about 50 mg/kg and for example from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

The compounds of the invention can also be administered topically, such as directly to the eye, e.g., as an eye-drop or ophthalmic ointment. Eye drops typically comprise an effective amount of at least one compound of the invention and a carrier capable of being safely applied to an eye. For example, the eye drops are in the form of an isotonic solution, and the pH of the solution is adjusted so that there is no irritation of the eye. In many instances, the epithelial barrier interferes with penetration of molecules into the eye. Thus, most currently used ophthalmic drugs are supplemented with some form of penetration enhancer. These penetration enhancers work by loosening the tight junctions of the most superior epithelial cells (Burstein, 1985, Trans Ophthalmol Soc U K 104(Pt 4): 402-9; Ashton et al., 1991, J Pharmacol Exp Ther 259(2): 719-24; Green et al., 1971, Am J Ophthalmol 72(5): 897-905). The most commonly used penetration enhancer is benzalkonium chloride (Tang et al., 1994, J Pharm Sci 83(1): 85-90; Burstein et al., 1980, Invest Ophthalmol Vis Sci 19(3): 308-13), which also works as preservative against microbial contamination. It is typically added to a final concentration of 0.01-0.05%.

In certain embodiments, the present invention is directed to a composition, as described herein, comprising a prodrug of a disclosed compound. The term “prodrug,” as used herein, means a compound that is convertible in vivo by metabolic means (e.g. by hydrolysis) to a compound. Various forms of prodrugs are known in the art such as those discussed in, for example, Bundgaard, (ed.), Design of Prodrugs, Elsevier (1985); Widder, et al. (ed.), Methods in Enzymology, vol. 4, Academic Press (1985); Krogsgaard-Larsen, et al., (ed). Design and Application of Prodrugs, Textbook of Drug Design and Development, Chapter 5, 113-191 (1991), Bundgaard, et al., Journal of Drug Delivery Reviews, 8:1-38(1992), Bundgaard, J. of Pharmaceutical Sciences, 77:285 et seq. (1988); and Higuchi and Stella (eds.) Prodrugs as Novel Drug Delivery Systems, American Chemical Society (1975), each of which is hereby incorporated by reference in its entirety.

For oral administration in the form of a tablet or capsule (e.g., a gelatin capsule), the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, magnesium aluminum silicate, starch paste, gelatin, methylcellulose, sodium carboxymethylcellulose and/or polyvinylpyrrolidone, natural sugars such as glucose or beta-lactose, corn sweeteners, natural and synthetic gums such as acacia, tragacanth or sodium alginate, polyethylene glycol, waxes and the like. Lubricants used in these dosage forms include sodium oleate, sodium stearate, magnesium stearate, sodium benzoate, sodium acetate, sodium chloride, silica, talcum, stearic acid, its magnesium or calcium salt and/or polyethyleneglycol and the like. Disintegrators include, without limitation, starch, methyl cellulose, agar, bentonite, xanthan gum starches, agar, alginic acid or its sodium salt, or effervescent mixtures, croscarmellose or its sodium salt, and the like. Diluents, include, e.g., lactose, dextrose, sucrose, mannitol, sorbitol, cellulose and/or glycine.

Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A therapeutically effective dose, of a compound described herein in an oral formulation, may vary from 0.01 mg/kg to 50 mg/kg patient body weight per day, more particularly 0.01 to 10 mg/kg, which can be administered in single or multiple doses per day. For oral administration, the drug can be delivered in the form of tablets or capsules containing 1 mg to 500 mg of the active ingredient specifically, 1 mg, 5 mg, 10 mg, 20 mg, 50 mg, 100 mg, 250 mg, and 500 mg, or in the forms of tables or capsules containing at least 1%, 2%, 5%, 10%, 15%, 20%, 25%, 30%, 40%, 50% (w/w) of the active ingredient. For example, the capsules may contain 50 mg of the active ingredient, or 5-10% (w/w) of the active ingredient. For example, the tablets may contain 100 mg of the active ingredient, or 20-50% (w/w) of the active ingredient. For example, the tablet may contain, in addition to the active ingredient, a disintegrant or emollient (e.g., croscarmellose or its sodium salt and methyl cellulose), a diluent (e.g., microcrystalline cellulose), and a lubricant (e.g., sodium stearate and magnesium stearate). The drug can be administered on a daily basis either once, twice or more per day.

For administration by inhalation, the compounds are delivered in the form of an aerosol spray from pressured container or dispenser, which contains a suitable propellant, e.g., a gas such as carbon dioxide, or a nebulizer.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

Parenteral formulations comprising a compound described herein can be prepared in aqueous isotonic solutions or suspensions, and suppositories are advantageously prepared from fatty emulsions or suspensions. The formulations may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional methods, and may contain about 0.1 to 75%, preferably about 1 to 50%, of a compound described herein.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

Formulations for topical administration to the skin can include, for example, ointments, creams, gels and pastes comprising the primary amine compound in a pharmaceutical acceptable carrier. The formulation of the primary amine compound for topical use includes the preparation of oleaginous or water-soluble ointment bases, as is well known to those in the art. For example, these formulations may include vegetable oils, animal fats, and, for example, semisolid hydrocarbons obtained from petroleum. Particular components used may include white ointment, yellow ointment, cetyl esters wax, oleic acid, olive oil, paraffin, petrolatum, white petrolatum, spermaceti, starch glycerite, white wax, yellow wax, lanolin, anhydrous lanolin and glyceryl monostearate. Various water-soluble ointment bases may also be used, including glycol ethers and derivatives, polyethylene glycols, polyoxyl 40 stearate and polysorbates.

The formulations for topical administration may contain the compound used in the present application at a concentration in the range of 0.001-10%, 0.05-10%, 0.1-10%, 0.2-10%, 0.5-10%, 1-10%, 2-10%, 3-10%, 4-10%, 5-10%, or 7-10% (weight/volume), or in the range of 0.001-2.0%, 0.001-1.5%, or 0.001-1.0%, (weight/volume), or in the range of 0.05-2.0%, 0.05-1.5%, or 0.05-1.0%, (weight/volume), or in the range of 0.1-5.0%, 0.1-2.0%, 0.1-1.5%, or 0.1-1.0% (weight/volume), or in the range of 0.5-5.0%, 0.5-2.0%, 0.5-1.5%, or 0.5-1.0% (weight/volume), or in the range of 1-5.0%, 1-2.0%, or 1-1.5% (weight/volume). The formulations for topical administration may also contain the compound used in the present application at a concentration in the range of 0.001-2.5%, 0.01-2.5%, 0.05-2.0%, 0.1-2.0%, 0.2-2.0%, 0.5-2.0%, or 1-2.0% (weight/weight), or in the range of 0.001-2.0%, 0.001-1.5%, 0.001-1.0%, or 0.001-5% (weight/weight).

### 5. Co-Administered Agents

In some embodiments, a second active pharmaceutical agent is co-administered with a compound or pharmaceutically acceptable salt thereof of the present invention.

In some embodiments, the compound above is used in combination with a second therapeutic agent. In some embodiments, the compounds of the disclosure can be administered with one or more of a second therapeutic agent, sequentially or concurrently, either by the same route or by different routes of administration. When administered sequentially, the time between administrations is selected to benefit, among others, the therapeutic efficacy and/or safety of the combination treatment. In some embodiments, the compound of the disclosure can be administered first followed by a second therapeutic agent, or alternatively, the second therapeutic agent administered first followed by the compound of the disclosure. In some embodiments, the compound of the disclosure can be administered for the same duration as the second therapeutic agent, or alternatively, for a longer or shorter duration as the second therapeutic compound.

When administered concurrently, the compounds of the disclosure can be administered separately at the same time as the second therapeutic agent, by the same or different routes, or administered in a single composition by the same route. In some embodiments, the compound of the disclosure is prepared as a first pharmaceutical composition, and the second therapeutic agent prepared as a second pharmaceutical composition, where the first pharmaceutical composition and the second pharmaceutical composition are administered simultaneously, sequentially, or separately. In some embodiments, the amount and frequency of administration of the second therapeutic agent can used standard dosages and standard administration frequencies used for the particular therapeutic agent. See, e.g., Physicians' Desk Reference, 70th Ed., PDR Network, 2015; incorporated herein by reference.

In some embodiments, the second therapeutic agent is non-steroidal anti-inflammatory drug (NSAID), antiviral agent, an antibiotic, or combinations thereof.

In some embodiments, the second therapeutic agent is an NSAID. In some embodiments, the NSAID is acetylsalicylic acid, diflunisal, salsalate, ibuprofen, dexibuprofen, naioxen, fenoprofen, ketoprofen, dexketoprofen, flurbiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lornoxicam, phenylbutazone, mefenamic acid, meclofenamic acid, flufenamic acid, tolfenamic acid, celecoxib or combinations thereof.

In some embodiments, the second therapeutic agent is an antiviral agent. In various embodiments, the antiviral agent is suitable for the viral infection associated with ARDS. In some embodiments, the second therapeutic agent is chloroquine, remdesivir, hydroxychloroquine, interferon, ribavirin, umifenovir, teicoplanin, lopinavir, ritonavir, didanosine, nitazoxanide, camostat, favipiravir, tocilizumab, or a passive antibody therapy.

In some embodiments, the second therapeutic agent is a passive antibody therapy. In some embodiments, passive antibody therapy uses blood donations from people who have recovered from COVID-19, a strategy that was tried for SARS. Antibodies produced by the immune systems of those who have already recovered are transferred to people in need of them via a nonvaccine form of immunization.

In some embodiments, the second therapeutic agent is

All features of each of the aspects of the invention apply to all other aspects mutatis mutandis.

### EXEMPLIFICATION

### Example 1: Pre-Clinical ARDS and Sepsis Models

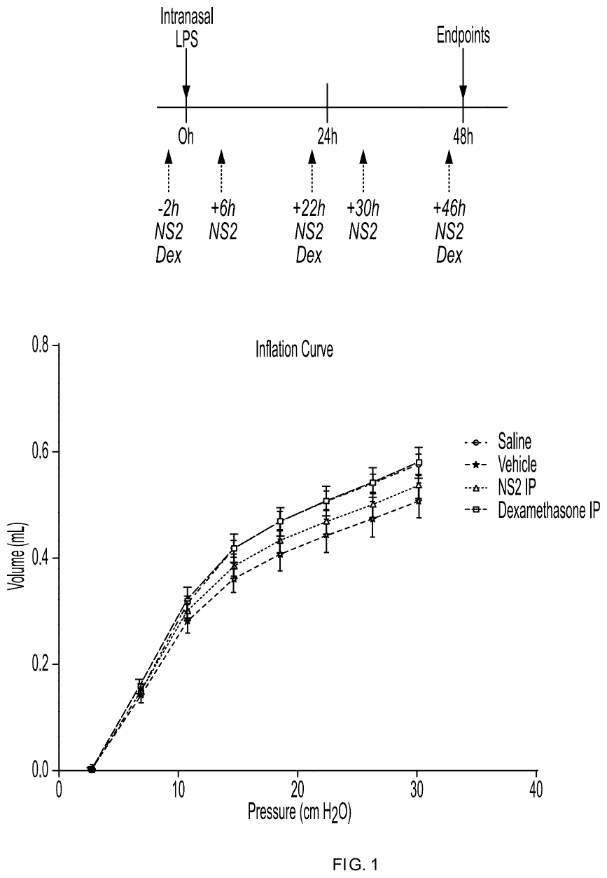

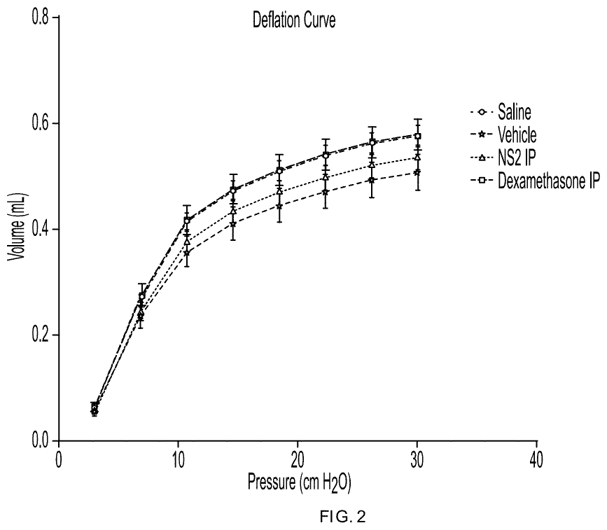

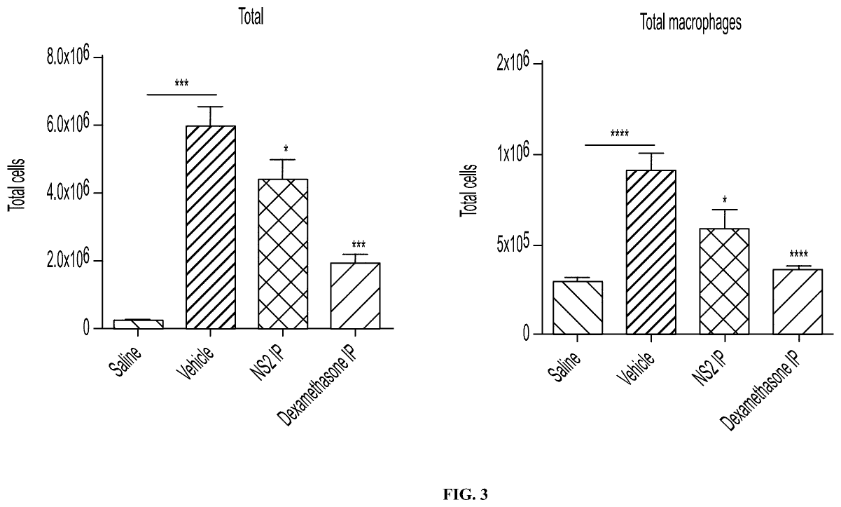

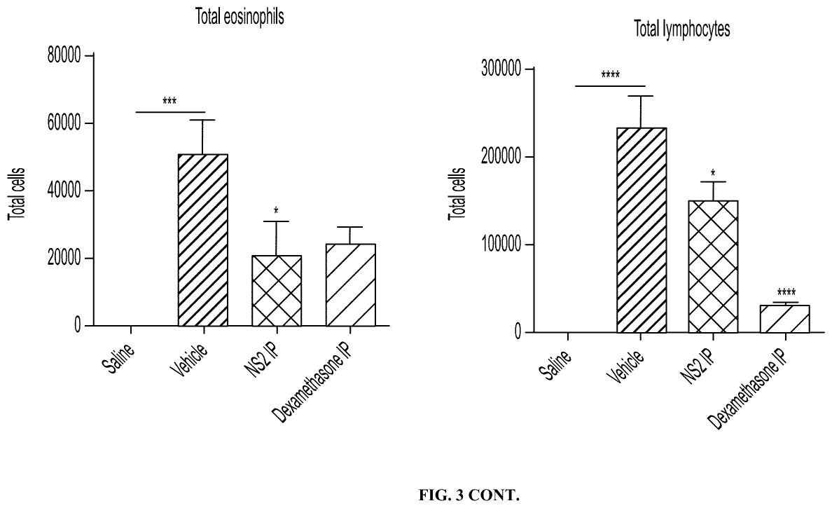

Free RASP are toxic, leading to inflammation and molecular dysfunction by covalently binding to cellular biomolecules, and have been linked to systemic immune-mediated and inflammatory diseases, such as NASH, inflammatory bowel disease (IBD), and other diseases. RASP inhibition with reproxalap (compound I-2, formerly known as NS2), a first-in-class RASP inhibitor, has been shown to have beneficial effects in treating ocular inflammation in clinical trials. I-2 showed promising activity in an LPS-induced acute lung injury model. FIG. 1 shows (top panel) a timeline of steps for a preclinical experiment in which NS2 (compound I-2) was compared with saline and dexamethasone (all administered IP) in an animal ARDS model. In the bottom panel, the effect of test compounds on leukocyte migration is shown in the model. FIG. 2 shows the effect of test compounds on leukocyte migration in a preclinical ARDS model. FIG. 3 shows the effect of test compounds on total cells, macrophages, total eosinophils, and lymphocytes in a preclinical ARDS model. I-2 decreased infiltrating cells and improved lung function in this model.

We have conducted in vivo studies to investigate how compound I-1 affects inflammatory pathways, and demonstrate that I-1 may have beneficial effects in systemic immune-mediated and inflammatory diseases. The anti-inflammatory effects of I-1 have been demonstrated in a lipopolysaccharide (LPS)-induced mouse model of acute sepsis, showing that I-1 can inhibit the release of a multitude of inflammatory cytokines and chemokines and facilitate the release of the anti-inflammatory cytokine, interleukin (IL)-10. These cytokines and chemokines affect Th1, Th2, and Th17 pathways, all of which are important in immune homeostasis and disease (Kaiko et al., Immunology 123:326-338, 2007. DOI 10.1111/j.1365-2567.2007.02719.x.; Weaver et al., Annu. Rev. Pathol 8:477-512, 2013. DOI 10.1146/annurev-pathol-011110-130318). In a dextran sulfate sodium (DSS)-induced mouse model of UC, I-1 improved several disease parameters, including body weight loss, disease activity score, colon length, colon edema, and colon inflammation. In addition, data from the three different rodent models of NASH provide evidence that I-1 may have beneficial effects on circulating markers of liver function and inflammation, including liver inflammation, steatosis, hepatocyte ballooning, and fibrosis.

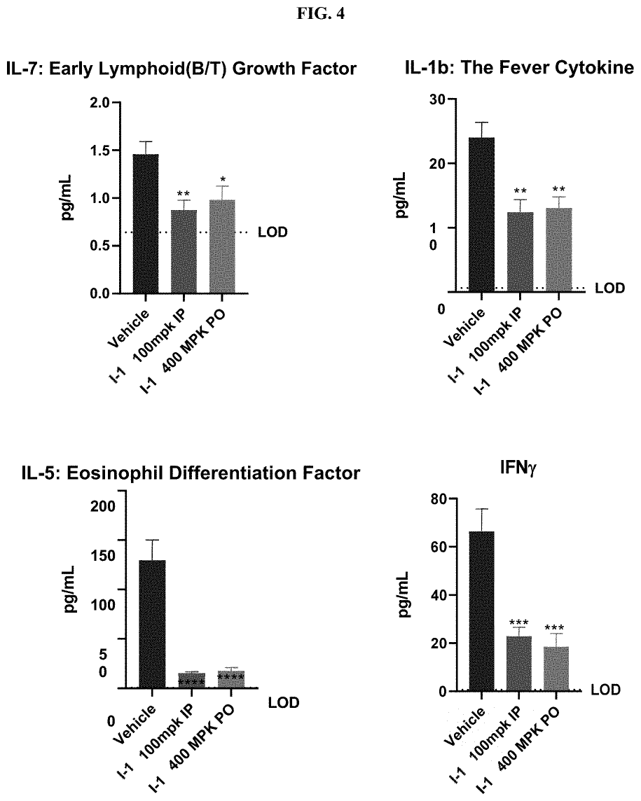

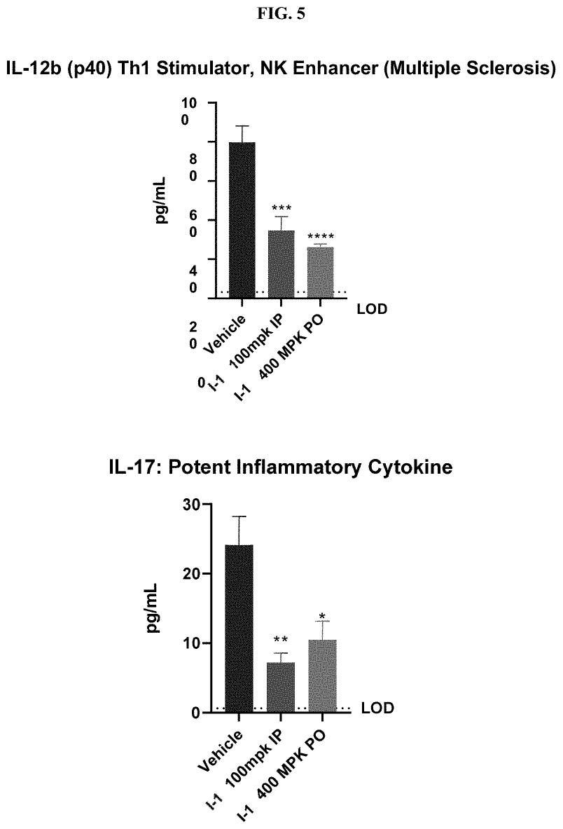

FIG. 4 and FIG. 5 show the effect of compound I-1 on levels of various inflammatory cytokines in a preclinical sepsis model. In the study, lipopolysaccharide (LPS) was administered IP to mice. 30 minutes later, I-1 was administered IP or PO. Mice were sacrificed after 2 hours, and their plasma submitted for cytokine profile analysis. IP=intraperitoneally; PO=orally. As shown in the table below, I-1 decreased several pro-inflammatory cytokines. * P≤0.05; ** P≤0.01; *** P≤0.001; **** P≤0.0001.

| | 100 mpk IP | 400 mpk PO |
|---|---|---|
| Description | Compound I-1 | Compound I-1 |
| Eotaxin | | |
| G-CSF | | |
| GM-CSF | * | |
| IFNγ | *** | *** |
| IL-1α | | |
| IL-1β | ** | ** |
| IL-2 | | |
| IL-3 | | |
| IL-4 | | |
| IL-5 | **** | **** |
| IL-6 | | |
| IL-7 | ** | * |
| IL-9 | | |
| IL-10 | ** | ** |
| IL-12 (p40) | *** | **** |
| IL-12 (p70) | | |
| IL-13 | | |
| IL-15 | | ** |
| IL-17 | ** | * |
| KC | | |
| LIF | | |
| LIX | | |
| MCP-1 | | ** |
| M-CSF | * | *** |
| MIP-1α | ** | * |
| MIP-1β | ** | * |
| MIP-2 | ** | |
| RANTES | | |
| TNFα | * | |
| VEGF | * | |

### Example 2: A Randomized, Double-Blind, Placebo-Controlled, Clinical Trial to Evaluate the Safety, Tolerability, Efficacy, and Pharmacodynamics of Compound I-1 Administered Orally for the Treatment of COVID-19

INDICATION: The indication for this Phase 2a clinical trial is the treatment of inflammation associated with coronavirus disease 2019 (COVID-19).

OBJECTIVES: The primary objective of this clinical trial is to evaluate the safety and tolerability of compound I-1, administered orally, in adult subjects with COVID-19 of moderate severity.

The secondary objectives of this clinical trial are the following:
To evaluate the National Institute of Allergy and Infectious Diseases (NIAID) 8-point ordinal scale for COVID-19 scores of adult subjects with COVID-19 of moderate severity who were administered compound I-1 orally; and
To assess the pharmacodynamics (PD) by measuring plasma reactive aldehyde species (RASP) in adult subjects with COVID-19 of moderate severity who were administered compound I-1 orally.

CLINICAL TRIAL DESIGN AND DURATION: Subjects are eligible for this trial if they are of 18 years of age or older with a documented, laboratory-confirmed severe acute respiratory syndrome coronavirus-2 (SARS-CoV-2) infection as determined by polymerase chain reaction, a SARS-CoV-2 antigen test, or another commercial or public health assay, within 3 days (72 hours) of randomization; and do not fall under any of the exclusion criteria.

This trial is a Phase 2a, randomized, double-blind, placebo-controlled, clinical trial to evaluate the safety, tolerability, efficacy, and PD of compound I-1, administered orally, in adult subjects with COVID-19 of moderate severity. Approximately 30 subjects will be randomized in a 2:1 ratio (compound I-1 to placebo) to receive either compound I-1 300 mg bis in die or twice daily (BID) or placebo BID for up to 28 days.

The reason for hospital admission (for hospitalized subjects), the standard of care followed for each subject and site, and whether any care decisions were based on resource limitations will be clearly documented for all subjects.

Treatment will begin on Day 1 following randomization. In addition to compound I-1 or placebo, all subjects will receive standard of care treatment for COVID-19. Treatment may commence whether a subject is hospitalized or is treated as an outpatient at Screening. During the Treatment Period (Days 1 to 28/End of Treatment [EOT]), all subjects will receive study drug (either I-1 or placebo) BID. The study drug will continue to be taken on an outpatient basis if a subject is discharged from the hospital prior to Day 28 for hospitalized subjects. If a subject is placed on mechanical ventilation and/or is otherwise unable to ingest an oral tablet, dosing will be stopped. However, the subject will continue to be monitored for efficacy and safety. Dosing of the study drug may resume once the subject is able to safely ingest an oral tablet.

All subjects will also have an assessment using the NIAID ordinal scale score daily on Days 1 to 28/EOT. The NIAID score will be evaluated directly for inpatient subjects and assessed via a subject diary for outpatient subjects through Day 28.

After treatment is completed, subjects will undergo a follow-up visit on Day 35 (+/−3 days) and a telephone follow-up on Day 60 (+/−3 days). The purpose of the telephone follow-up is to assess survival status and evaluate for COVID-19 relapse. A telephone follow-up visit may be performed in lieu of the Day 35 follow-up visit if the subject is unable to attend in person.

Dosage Forms and Route of Administration:

I-1 and the corresponding placebo will each be administered orally as a tablet without food, defined as no food for at least 2 hours prior to dosing and at least 1 hour after dosing. Each subject will receive either I-1 300 mg BID or placebo BID for up to 28 days. If a subject is temporarily unable to tolerate dosing, a temporary dose interruption of up to 2 days may be permitted. No dose reductions will be permitted.

I-1 and the corresponding placebo will each be available as a tablet for oral administration.

Endpoints: The primary endpoint is the safety of I-1, determined by evaluation of the following:
Adverse Events (AEs);
Safety laboratory tests (including chemistry, hematology, coagulation parameters, and urinalysis);
Vital signs (including heart rate, blood pressure, respiratory rate, temperature, and oxygen saturation);
Physical examinations; and
12-lead electrocardiograms.

The key secondary endpoint is the NIAID ordinal scale score over Day 1 through Day 28. Additional secondary endpoints include the following:
Proportion of subjects alive and not mechanically ventilated over Day 1 through Day 28;
Time to recovery (for hospitalized subjects at Screening), defined as the first day on which the subject satisfies 1 of the following 3 categories from the NIAID ordinal scale:
   Hospitalized, not requiring supplemental oxygen—no longer requires ongoing medical care;
   Not hospitalized, limitation on activities and/or requiring home oxygen; or
   Not hospitalized, no limitations on activities;
Time to recovery (for non-hospitalized subjects at Screening), defined as the second consecutive day on which the subject has an improvement in the NIAID ordinal scale by 1 point (e.g., NIAID scale 7 to 8);
Proportion of subjects alive and not in the intensive care unit over Day 1 through Day 28;
Proportion of subjects alive and not in the hospital over Day 1 through Day 28;
Proportion of subjects alive and not on supplemental oxygen over Day 1 through Day 28;
Mortality; and
Time to hospital discharge (for hospitalized subjects).

Pharmacodynamic Endpoints:

The PD endpoints will include the following:
RASP plasma concentrations; and
Plasma exploratory biomarker concentrations (e.g., interleukin [IL]-1β, IL-6, IL-10, and tumor necrosis factor alpha).
   RASP species to be measured include malondialdehyde and 4-hydroxynonenal.

Statistical Analyses:

The following analysis populations are defined for this clinical trial:
Intent-to-Treat (ITT) Population: All randomized subjects;
Safety Population: All randomized subjects who receive at least 1 dose of study drug;
Per-Protocol Population: All subjects in the ITT Population without major protocol deviations; and
PD Population: All subjects who have at least 1 PD measurement.

Clinical parameters will be assessed via linear models with repeated measures over 28 days. Sensitivity analyses will be performed using other techniques.

INTRODUCTION AND BACKGROUND: The target population in this clinical trial is adult subjects with coronavirus disease 2019 (COVID-19) of moderate severity. It is postulated that I-1 may mitigate the inflammatory response sufficiently in these subjects to lower levels of inflammatory mediators and potentially improve clinical outcomes.

Coronaviruses (CoV) are common causes of respiratory and gastrointestinal infections in a variety of animal species, including humans (Reference 1). Numerous strains of CoV have been discovered, 7 of which have been identified to infect humans. Most of these strains cause mild respiratory symptoms in immunocompetent hosts; however, 3 strains, including the novel severe acute respiratory syndrome (SARS)-CoV-2 (SARS-CoV-2), cause severe respiratory syndromes. The SARS-CoV-1, Middle East respiratory syndrome (MERS)—CoV, and SARS-CoV-2 can all result in severe pneumonia and acute respiratory distress syndrome (ARDS), a frequently fatal condition (References 1, 2).

Although fatality rates from SARS-CoV-1 and MERS-CoV, 9.5% and 34.4% respectively (Reference 2), are higher than reported rates for SARS-CoV-2 (ranging from ≤1.0% to over 6% [as of 31 May 2020]; References 3, 4), SARS-CoV-2 is more easily transmitted. SARS-CoV-2 originated in December 2019 in Wuhan, China. Secondary to its high reproductive number, SARS-CoV-2 quickly spread across China and the world despite attempts at quarantining, social distancing, and using personal protective measures. The World Health Organization declared the SARS-CoV-2 and resulting COVID-19 as a pandemic and public health emergency of international concern on 30 Jan. 2020 (Reference 5).

The COVID-19 pandemic has led to an urgent need for new therapies. Many patients diagnosed with COVID-19 will develop ARDS and require ventilatory support. In general, ARDS is associated with mortality rates up to 40% (Reference 6); however, mortality from COVID-19-induced ARDS is even higher, with reported death rates over 50% (References 7, 8, 9, 10).

The worst outcomes are observed in older patients and those with chronic diseases (Reference 7). A treatment that could prevent the progression to severe COVID-19 could potentially reduce morbidity and mortality from the disease.

CLINICAL TRIAL TREATMENTS: All subjects will receive either I-1 300 mg BID or placebo BID for up to 28 days. The study drug will be administered orally as a tablet.

Excluded Medications and/or Procedures: The following medications are prohibited:
Systemic immunomodulatory therapy (e.g., corticosteroids, calcineurin inhibitors, hydroxychloroquine, anti-cytokine therapies, Janus kinase inhibitors) except for daily prednisone (or steroid equivalent)≤10 mg or inhaled or nasal corticosteroids;
Note: Dexamethasone 6 mg daily or equivalent is permitted after randomization for progression to severe or critical COVID-19 requiring invasive ventilation or oxygen therapy for hypoxemia.
Duloxetine, bupropion, statins, phosphodiesterase inhibitors, midazolam, fluoxetine, fluvoxamine, fluconazole, or ciprofloxacin; another CYP1A2, 2B6, or 3A4 sensitive substrate; or a CYP1A2, 2C19, or 3A4 inhibitor that would result in either supratherapeutic or subtherapeutic drug levels, placing the subject at undue risk by participating in the clinical trial;
Acetaminophen use >2 g in 24 hours; and
Investigational products, other than the study drug.

Procedure During Treatment Period (Days 1 Through 28/EOT): During the Treatment Period, all subjects will receive study drug (either I-1 or placebo) BID on Days 1 to 28/EOT. Treatment may commence whether a subject is hospitalized or is treated as an outpatient at Screening. The study drug will continue to be taken on an outpatient basis if a subject is discharged from the hospital prior to Day 28 for hospitalized subjects. The study drugs will each be administered orally BID without food, defined as no food for at least 2 hours prior to dosing and at least 1 hour after dosing. Inpatient subjects will receive the study drug in the hospital. Outpatient subjects will take the study drug at home according to their usual schedule, including on Days 1, 7, 13, 22, and 28 when outpatient subjects have onsite visits.

All subjects will also have an assessment using the NIAID ordinal scale score daily on Days 1 to 28/EOT. Inpatient subjects will have an assessment of standard of care daily on Days 1 to 28/EOT (or until discharge).

SAFETY ASSESSMENTS: The safety of I-1 will be determined by evaluation of the following:
AEs;
Safety laboratory tests (including chemistry, hematology, coagulation parameters, and urinalysis);
Vital signs (including heart rate, blood pressure, respiratory rate, temperature, and oxygen saturation);
Physical examinations; and
12-lead ECGs.

FDA Examples of Baseline Severity Categorization. SARS-CoV-2 infection without symptoms:
Positive testing by standard reverse transcription polymerase chain reaction (RT-PCR) assay or equivalent test; and
No symptoms.

Mild COVID-19:
Positive testing by standard RT-PCR assay or equivalent test;
Symptoms of mild illness with COVID-19 that could include fever, cough, sore throat, malaise, headache, muscle pain, or gastrointestinal symptoms, without shortness of breath or dyspnea; and
No clinical signs indicative of Moderate, Severe, or Critical severity.

Moderate COVID-19
Positive testing by standard RT-PCR assay or equivalent test;
Symptoms of moderate illness with COVID-19, which could include any symptom of mild illness or shortness of breath with exertion;
Clinical signs suggestive of moderate illness with COVID-19, such as respiratory rate
   20 breaths per minute, saturation of oxygen >93% on room air at sea level, or heart rate
   90 beats per minute; and
   No clinical signs indicative of Severe or Critical severity.

Severe COVID-19:
Positive testing by standard RT-PCR assay or equivalent test;
Symptoms suggestive of severe systemic illness with COVID-19, which could include any symptom of moderate illness or shortness of breath at rest, or respiratory distress;
Symptoms suggestive of severe systemic illness with COVID-19, such as respiratory rate 30 breaths per minute, saturation of oxygen ≤93% on room air at sea level, heart rate 125 beats per minute, or partial pressure of oxygen/fraction of inspired oxygen ≤300; and
No criteria for Critical severity.

Critical COVID-19 is defined as:
Positive testing by standard RT-PCR assay or equivalent test; and
Evidence of critical illness, defined by at least 1 of the following:
   Respiratory failure defined based on resource utilization requiring at least 1 of the following:
      Endotracheal intubation and mechanical ventilation, oxygen delivered by high-flow nasal cannula (heated, humidified, oxygen delivered via reinforced nasal cannula at flow rates >20 L/minute with fraction of delivered oxygen ≥0.5), noninvasive positive pressure ventilation, extracorporeal membrane oxygenation, or clinical diagnosis of respiratory failure (i.e., clinical need for 1 of the preceding therapies, but preceding therapies are not able to be administered in setting of resource limitation);
   Shock (defined by systolic blood pressure ≤90 mmHg, diastolic blood pressure ≤60 mmHg, or requiring vasopressors); or
   Multi-organ dysfunction/failure.
      Source: US Department of Health and Human Services, Food and Drug Administration, Center for Drug Evaluation and Research (CDER), Center for Biologics Evaluation and Research (CBER). Guidance for industry: COVID-19: Developing drugs and biological products for treatment or prevention. May 2020.

### REFERENCES

1. Cui J, Li F, Shi Z L. Origin and evolution of pathogenic coronaviruses. Nat Rev Microbiol. 2019; 17(3): 181-192.
2. Petrosillo N, Viceconte G, Ergonul O, et al. COVID-19, SARS, and MERS: are they closely related? Clin Microbiol Infect. 2020; 26(6):729-734.
3. Centers for Disease Control and Prevention. Coronavirus 2019 (COVID-19): COVID-19 pandemic planning scenarios. Centers for Disease Control and Prevention website. cdc.gov/coronavirus/2019-ncov/hcp/planning-scenarios.html. Accessed 31 May 2020.
4. World Health Organization. WHO coronavirus disease (COVID-19) dashboard. World Health Organization website. covid19.who.int/. Accessed 31 May 2020.
5. World Health Organization. Statement on the second meeting of the International Health Regulations (2005) Emergency Committee regarding the outbreak of novel coronavirus (2019-nCoV). News release. 30 Jan. 2020. who.int/news-room/detail/30-01-2020-statement-on-the-second-meeting-of-the-international-health-regulations(2005)-emergency-committee-regarding-the-outbreak-of-novel-coronavirus-(2019-ncov). Accessed 27 May 2020.
6. Walkey A J, Summer R, Ho V, et al. Acute respiratory distress syndrome: epidemiology and management approaches. Clin Epidemiol. 2012; 4(1):159-169.
7. Wu C, Chen X, Cai Y, et al. Risk factors associated with acute respiratory distress syndrome and death in patients with coronavirus disease 2019 pneumonia in Wuhan, China. JAMA Intern Med. 2020. doi: 10.1001/jamainternmed.2020.0994. Accessed 27 May 2020.
8. Chen N, Zhou M, Dong X, et al. Epidemiological and clinical characteristics of 99 cases of 2019 novel coronavirus pneumonia in Wuhan, China: a descriptive study. Lancet. 2020; 395(10223):507-513.
9. Bhatraju P K, Ghassemieh B J, Nichols M, et al. Covid-19 in critically ill patients in the Seattle region—case series. N Engl J Med. 2020. doi: 10.1056/NEJMoa2004500. Accessed 27 May 2020.
10. Arentz M, Yim E, Klaff L. Characteristics and outcomes of 21 critically ill patients with COVID-19 in Washington State. JAMA. 2020; 323(16):1612-1614.
11. Kaiko G E, Horvat J C, Beagley K W, et al. Immunological decision-making: how does the immune system decide to mount a helper T-cell response? Immunology. 2008; 123(3):326-338.
12. Weaver C T, Elson C O, Fouser L A, et al. The Th17 pathway and inflammatory diseases of the intestines, lungs, and skin. Annu Rev Pathol. 2013; 8:477-512.

We claim:

1. A method of treating acute respiratory distress syndrome (ARDS), comprising administering to a patient in need thereof an effective amount of a compound of Formula I: 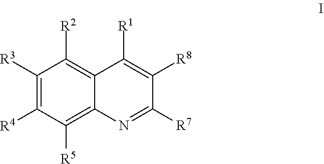
or a pharmaceutically acceptable salt thereof, wherein:
each of R¹, R⁷, and R⁸ is independently H, D, halogen, —NH₂, —CN, —OR, —SR, optionally substituted C₁₋₆ aliphatic, or 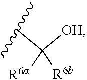
wherein one of R¹, R⁷, and R⁸ is —NH₂ and one of R¹, R⁷, and R⁸ is 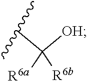
R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{6a} is C₁₋₄ aliphatic optionally substituted with 1, 2, or 3 deuterium or halogen atoms;
R^{6b} is C₁-4 aliphatic optionally substituted with 1, 2, or 3 deuterium or halogen atoms; or R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

2. A method of treating acute respiratory distress syndrome (ARDS), comprising administering to a patient in need thereof an effective amount of a compound of Formula II: 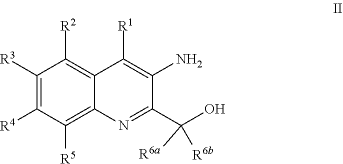
or a pharmaceutically acceptable salt thereof, wherein:
R¹ is H, D, or halogen;
R² is H, D, or halogen;
R³ is H, D, or halogen;
R⁴ is H, D, or halogen;
R⁵ is H, D, or halogen;
R^{6a} is C₁₋₄ aliphatic optionally substituted with 1, 2, or 3 deuterium or halogen atoms; and
R^{6b} is C₁₋₄ aliphatic optionally substituted with 1, 2, or 3 deuterium or halogen atoms.

3. The method of claim 1 or 2, wherein R^{6a} and R^{6b} are methyl or ethyl optionally substituted with 1, 2, or 3 deuterium or halogen atoms.

4. The method of claim 1 or 2, wherein R^{6a} and R^{6b} are methyl.

5. The method of claim 1, wherein the compound is selected from: 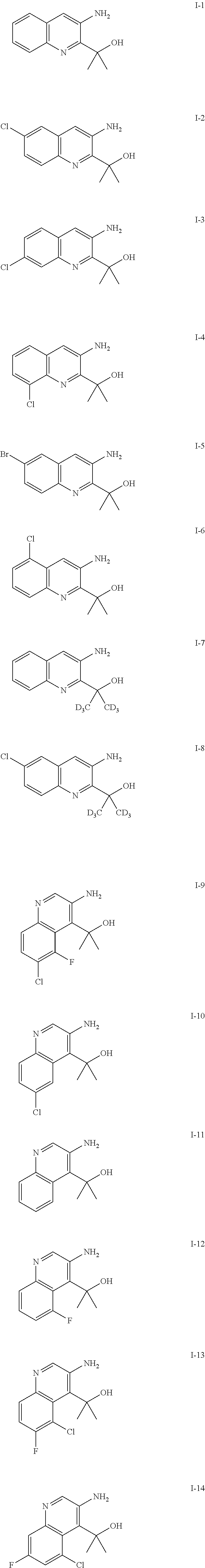 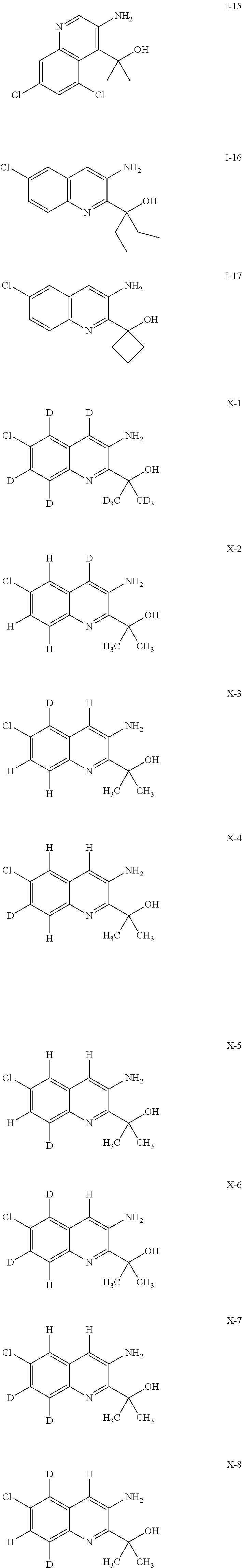 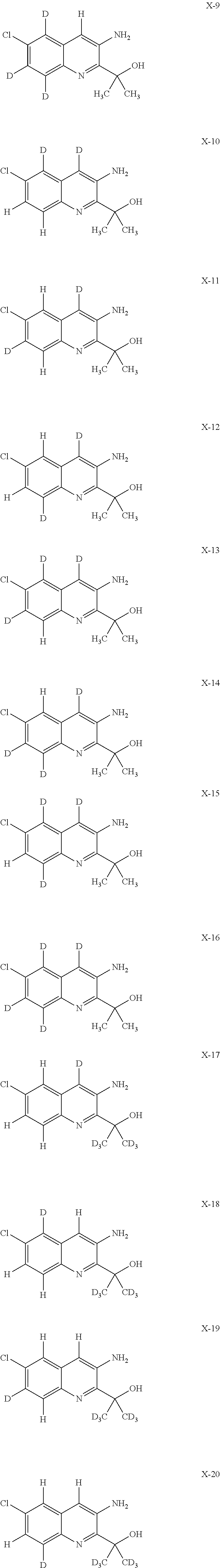 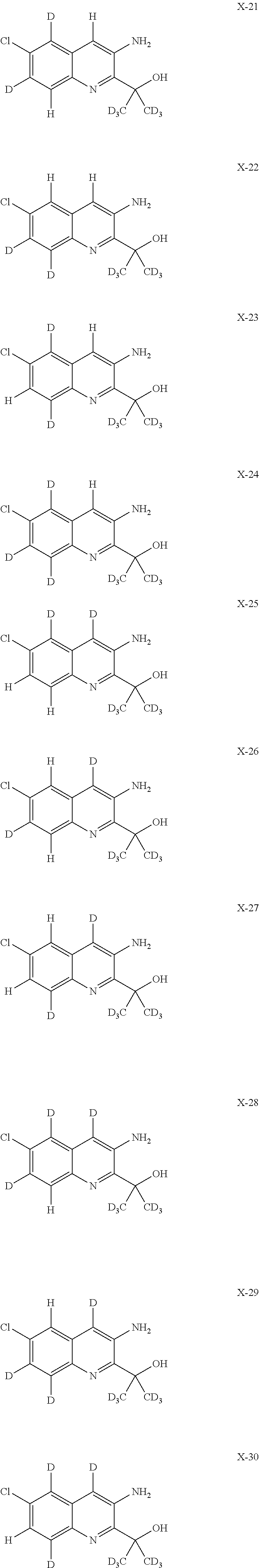
or a pharmaceutically acceptable salt thereof.

6. The method of claim 1 or 2, wherein the compound is 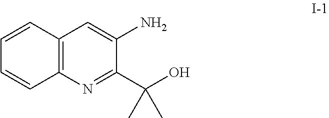
or a pharmaceutically acceptable salt thereof.

7. The method of claim 1 or 2, wherein the compound is 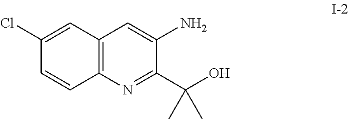 [SMILES: CC(C)(O)c1nc2ccc(Cl)cc2cc1N]
or a pharmaceutically acceptable salt thereof.

8. The method of any one of claims 1-7, wherein the ARDS is associated with a viral infection.

9. The method of claim 8, wherein the viral infection manifests as viral sepsis.

10. The method of claim 8 or 9, wherein the viral infection is accompanied by viral pneumonia.

11. The method of any one of claims 8-10, wherein the viral infection is by a respiratory syncytial virus (RSV), influenza virus, coronavirus, or herpesvirus.

12. The method of claim 11, wherein the viral infection is by a coronavirus.

13. The method of claim 12, wherein the coronavirus is selected from 229E, NL63, OC43, HKU1, MERS-CoV, SARS-CoV, and SARS-CoV-2.

14. The method of claim 13, wherein the viral infection is by SARS-CoV-2.

15. The method of claim 11, wherein the viral infection is by an influenza virus.

16. The method of claim 15, wherein the influenza virus is influenza type A or influenza type B.

17. The method of claim 16, wherein the influenza virus is B/Yamagata or B/Victoria.

18. The method of claim 16, wherein the influenza virus is H5N1, H1N1 or H3N2.

19. The method of any one of claims 1-7, wherein the ARDS is associated with a bacterial infection.

20. The method of claim 19, wherein the bacterial infection is accompanied by bacterial sepsis.

21. The method of claim 19 or 20, wherein the bacterial infection is accompanied by bacterial pneumonia.

22. The method of any one of claims 19 to 21, wherein the bacterial infection is by Streptococcus pneumoniae, Staphylococcus aureus, Legionella pneumophila, Pneumocystis jirovecii, or Haemophilus influenza.

23. The method of any one of claims 1-7, wherein the ARDS is associated with acute injury to the lungs caused by a chemical toxin or physical trauma.

24. The method of claim 23, wherein the acute injury to the lungs is by a chemical toxin.

25. The method of claim 24, wherein the chemical toxin that causes acute lung injury is a choking agent, vesicant, or nerve agent.

26. The method of claim 25, wherein the chemical toxin is a choking agent, wherein the choking agent is chlorine gas, phosgene, carbonyl chloride, hydrogen sulfide, or ammonia.

27. The method of claim 25, wherein the chemical toxin is a vesicant, wherein the vesicant is sulfur mustard or nitrogen mustard.

28. The method of claim 25, wherein the chemical toxin is a nerve agent, wherein the nerve agent is tabun, sarin, soman, or VX.

29. The method of any one of claims 1-7, wherein the ARDS is associated with acute injury to the lungs caused by a biological toxin.

30. The method of claim 29, wherein the biological toxin is ricin, botulinum toxin, or staphylococcal enterotoxin B.

31. The method of any one of claims 1-30, wherein the patient is being treated by mechanical ventilation.

32. The method of any one of claims 1-31, wherein the dose of the compound or pharmaceutically acceptable salt thereof is about 400 mg to about 1200 mg per day.

33. The method of any one of claims 1-31, wherein the dose of the compound or pharmaceutically acceptable salt thereof is about 50 mg to about 3600 mg per day.

34. The method of any one of claims 1-31, wherein the dose of the compound or pharmaceutically acceptable salt thereof is about 250 mg to about 2400 mg per day.

35. The method of any one of claims 1-34, wherein the dose of the compound or pharmaceutically acceptable salt thereof is administered systemically.

36. The method of any one of claims 1-34, wherein the dose of the compound or pharmaceutically acceptable salt thereof is administered orally.

37. The method of any one of claims 1-36, further comprising administering to the patient an effective amount of a second therapeutic agent suitable for treating ARDS.

38. The method of claim 37, wherein the anti-inflammatory agent is a steroid, anti-GM-CSF antibody, or lung surfactant.

39. A method of treating a viral infection, comprising administering to a patient in need thereof an effective amount of a compound of Formula I: 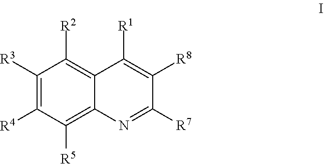
or a pharmaceutically acceptable salt thereof, wherein:
each of R¹, R⁷, and R⁸ is independently H, D, halogen, —NH₂, —CN, —OR, —SR, optionally substituted C₁₋₆ aliphatic, or 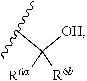
wherein one of R¹, R⁷, and R⁸ is —NH₂ and one of R¹, R⁷, and R⁸ is 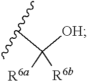
R² is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R³ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁴ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R⁵ is selected from —R, halogen, —CN, —OR, —SR, —N(R)₂, —N(R)C(O)R, —C(O)N(R)₂, —N(R)C(O)N(R)₂, —N(R)C(O)OR, —OC(O)N(R)₂, —N(R)S(O)₂R, —SO₂N(R)₂, —C(O)R, —C(O)OR, —OC(O)R, —S(O)R, and —S(O)₂R;
R^{6a} is C₁₋₄ aliphatic optionally substituted with 1, 2, or 3 deuterium or halogen atoms;
R^{6b} is C₁₋₄ aliphatic optionally substituted with 1, 2, or 3 deuterium or halogen atoms; or R^{6a} and R^{6b}, taken together with the carbon atom to which they are attached, form a 3- to 8-membered cycloalkyl or heterocyclyl ring containing 1-2 heteroatoms selected from nitrogen, oxygen, and sulfur; and
each R is independently selected from hydrogen, deuterium, and an optionally substituted group selected from C₁₋₆ aliphatic; a 3- to 8-membered saturated or partially unsaturated monocyclic carbocyclic ring; phenyl; an 8- to 10-membered bicyclic aryl ring; a 3- to 8-membered saturated or partially unsaturated monocyclic heterocyclic ring having 1-4 heteroatoms independently selected from nitrogen, oxygen, and sulfur; a 5- to 6-membered monocyclic heteroaryl ring having 1-4 heteroatoms independently selected from nitrogen, oxygen, and sulfur; a 6- to 10-membered bicyclic saturated or partially unsaturated heterocyclic ring having 1-5 heteroatoms independently selected from nitrogen, oxygen, and sulfur; and a 7- to 10-membered bicyclic heteroaryl ring having 1-5 heteroatoms independently selected from nitrogen, oxygen, and sulfur.

40. A method of treating a viral infection, comprising administering to a patient in need thereof an effective amount of a compound of Formula II: 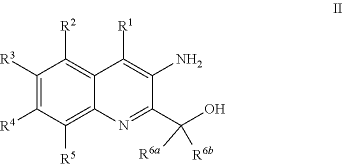
or a pharmaceutically acceptable salt thereof, wherein:
R¹ is H, D, or halogen;
R² is H, D, or halogen;
R³ is H, D, or halogen;
R⁴ is H, D, or halogen;
R⁵ is H, D, or halogen;
R^{6a} is C₁₋₄ aliphatic optionally substituted with 1, 2, or 3 deuterium or halogen atoms; and
R^{6b} is C₁₋₄ aliphatic optionally substituted with 1, 2, or 3 deuterium or halogen atoms.

41. The method of claim 39 or 40, wherein R^{6a} and R^{6b} are methyl or ethyl optionally substituted with 1, 2, or 3 deuterium or halogen atoms.

42. The method of claim 39 or 40, wherein R^{6a} and R^{6b} are methyl.

43. The method of claim 39, wherein the compound is selected from: 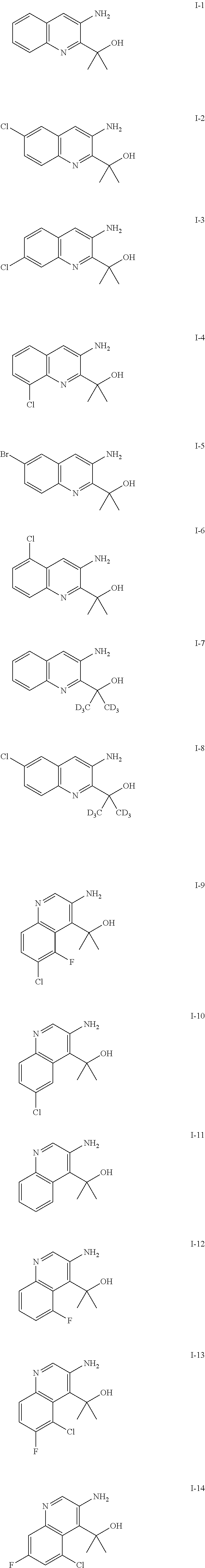 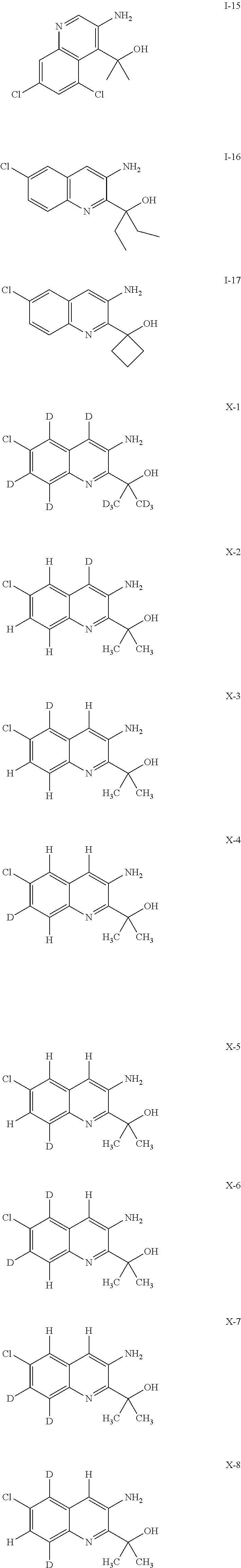 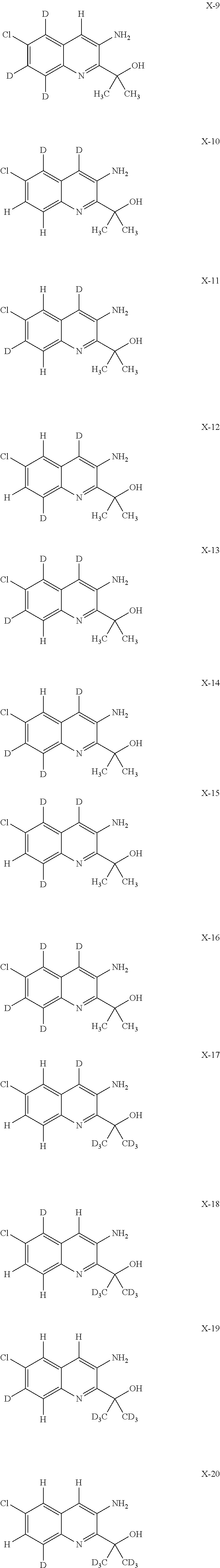 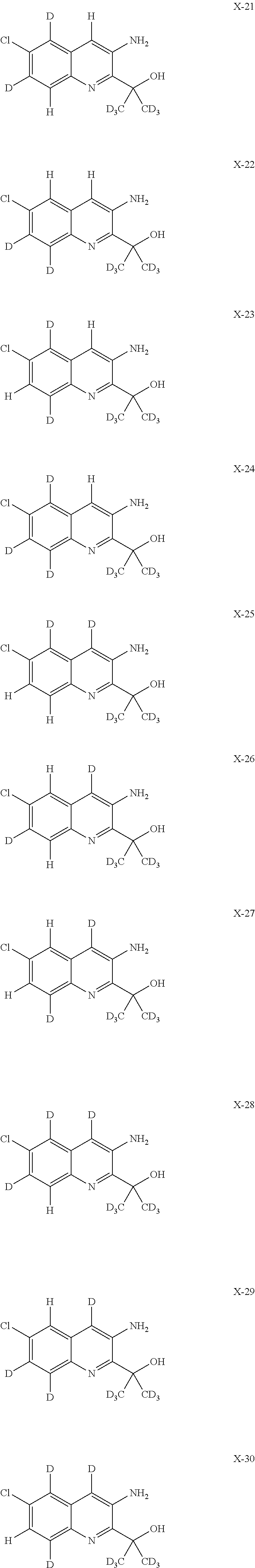
or a pharmaceutically acceptable salt thereof.

44. The method of claim 39 or 40, wherein the compound is 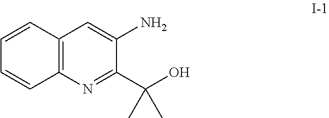
or a pharmaceutically acceptable salt thereof.

45. The method of claim 39 or 40, wherein the compound is 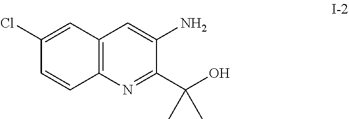 [SMILES: CC(C)(O)c1nc2ccc(Cl)cc2cc1N]
or a pharmaceutically acceptable salt thereof.

46. The method of any one of claims 39-44, wherein the viral infection is caused by a coronavirus, hepatitis A virus, hepatitis B virus, dengue virus, yellow fever virus, Zika virus, influenza virus, norovirus, herpesvirus, respiratory syncytial virus (RSV), human immunodeficiency virus (HIV), Ebola virus, human T-lymphotropic virus (HTLV)-1 and -2, Epstein-Barr virus, Lassa virus, or Crimean-Congo hemorrhagic fever virus.

47. The method of claim 45, wherein the viral infection is caused by a coronavirus.

48. The method of claim 46, wherein the viral infection is caused by a coronavirus selected from 229E, NL63, OC43, HKU1, MERS-CoV, SARS-CoV, and SARS-CoV-2.

49. The method of claim 47, wherein the viral infection is caused by SARS-CoV-2.

50. The method of claim 45, wherein the viral infection is caused by an influenza virus.

51. The method of claim 45, wherein the viral infection is caused by an influenza virus selected from influenza type A and influenza type B.

52. The method of claim 45, wherein the viral infection is caused by an influenza virus selected from H5N1, H1N1 and H3N2.

53. The method of claim 45, wherein the viral infection is caused by the Zika virus.

54. The method of any one of claims 39-53, wherein the dose of the compound or pharmaceutically acceptable salt thereof is about 400 mg to about 1200 mg per day.

55. The method of any one of claims 39-53, wherein the dose of the compound or pharmaceutically acceptable salt thereof is about 50 mg to about 3600 mg per day.

56. The method of any one of claims 39-53, wherein the dose of the compound or pharmaceutically acceptable salt thereof is about 250 mg to about 2400 mg per day.

57. The method of any one of claims 39-55, wherein the dose of the compound or pharmaceutically acceptable salt thereof is administered systemically.

58. The method of any one of claims 39-56, wherein the dose of the compound or pharmaceutically acceptable salt thereof is administered orally.

59. The method of any one of claims 39-57, further comprising administration of a second therapeutic agent selected from an antiviral agent, an antibiotic, a steroid, a monoclonal antibody, convalescent plasma, and an NSAID.

60. The method of claim 58, wherein the second therapeutic agent is an antiviral agent, wherein the antiviral agent is appropriate for treating the viral infection.

61. The method of claim 59, further comprising administering a second therapeutic agent selected from chloroquine, remdesivir, hydroxychloroquine, interferon, ribavirin, umifenovir, teicoplanin, lopinavir, ritonavir, nitazoxanide, camostat, favipiravir, tocilizumab, and a passive antibody therapy.
